(19) United States
(12) Patent Application Publication
(10) Pub. No.: US 2019/0255138 A1
(43) Pub. Date: Aug. 22, 2019

(54) A NATURAL COMPOSITION TO INHIBIT BETA-SECRETASE AND ENHANCE BIOAVAILABILITY COMPRISING OF TURMERIC AND SESAME SEED EXTRACT

(71) Applicant: Benny ANTONY, ANGAMALY (IN)

(72) Inventor: Benny ANTONY, ANGAMALY (IN)

(21) Appl. No.: 16/315,503

(22) PCT Filed: Jul. 6, 2017
(86) PCT No.: PCT/IB2017/054072

§ 371 Date: Jan. 4, 2019

(30) Foreign Application Priority Data
Jul. 8, 2016 (IN) 201641023403

(51) Int. Cl. A61K 36/9066; A61K 31/121; A61K 36/185; A61K 9/14; A61P 25/28; A61P 25/08; A61P 25/16; A61P 25/24

(52) U.S. Cl. CPC A61K 36/9066; A61K 31/121; A61K 36/185; A61K 9/14; A61P 25/28; A61P 25/08; A61K 2236/39; A61P 25/24; A61K 2236/10; A61K 2236/55; A61K 2236/333; A61K 2236/35; A61P 25/16

(57) ABSTRACT

Compositions for inhibiting beta secretase activity are disclosed. Compositions that enhance bioavailability of curcumin, demethoxycurcumin, bisdemethoxycurcumin, α-turmerone, ar-turmerone, or, sesamolin are disclosed. Some compositions include a curcuminoid mixture and essential oil of turmeric having α-turmerone. Some compositions include a curcuminoid mixture and a combination, wherein the combination includes essential oil of turmeric having α-turmerone, and, an extract of sesame oil having sesamolin. Some compositions are a combination of essential oil of turmeric having α-turmerone, and, an extract of sesame oil having sesamolin. Methods of preparing the compositions are disclosed. Methods of treatment of central nervous system or neurological disorders are provided.

### FIELD OF INVENTION

The invention pertain to a medicinal composition comprising of essential oil of turmeric with α-turmerone along with curcuminoid or sesame seed extract enriched with sesamolin or both in a specific ratio which is capable of inhibiting f-secretase and enhance bioavailability. More particularly said composition enhances the curcuminoid, α-turmerone and sesamolin bioavailability in brain and contains curcuminoid, purified essential oil of turmeric and sesame seed extract enriched with sesamolin. The curcuminoid mixture includes curcumin, demethoxycurcumin, and, bisdemethoxycurcumin. The essential oil of turmeric includes about 30% to about 80% of α-turmerone. The extract of sesame oil includes about 30% to about 80% sesamolin.

Further a process of extraction of curcuminoid from turmeric, in addition to a process of extraction of α-turmerone from turmeric and enrichment of α-turmerone to obtain a purity of 80% is also disclosed. Similarly a process of extraction of sesamolin from sesame seed are also disclosed.

The disclosure provides a method of treatment of neurological disorder by orally administering a blend of curcuminoids with purified essential oil of turmeric enriched with α-turmerone and enriched sesamolin from sesame seed extract.

### BACKGROUND

Nowadays the use of botanical products in food and medicine is on the increase. This has led to an intense research in to plant extracts and their health benefits. Traditionally known benefits of plants are being analysed and validated for pharmacological benefits. At the same time they are compared against modern pharmaceutical drugs for their efficacy. One of the well known plants, extensively used in food and nutraceutical industry is turmeric. Turmeric (Curcuma longa) is a member of the ginger family (Zingiberaceae). It is a perennial rhizomatous herbaceous plant native to southern Asia, extensively cultivated in all parts of India. Turmeric is extensively used in south Asian cuisine, particularly in India. Turmeric is also used for its medicinal properties in traditional remedies, particularly in Ayurveda, an Indian traditional medical system. Turmeric in combination with other herbs is used for, but not limited to the following: internally as a tonic, blood purifier, and externally prevention and treatment of skin diseases. Turmeric also has a long history of use for its anti-inflammatory and anti-arthritic effects. Scientific research into biologically active compounds in turmeric has shown many health benefits including free radical scavenging and anti-cancer activity. It decreases symptoms of skin cancers and reduces the incidence of chemically caused breast cancer in animals.

The most active components of turmeric are the curcuminoids. They are the primary non volatile components of turmeric. Curcumin which make up for about 80% of the curcuminoids is the most studied component of turmeric which is also responsible for giving turmeric its unique yellow colour. Curcumin is known for its anti-inflammatory and antioxidant activities, curcumin has also shown activities to reduce oxidative damage related to aging. Aiguo et al. (2005) have shown how curcumin can reduce oxidative stress in brain, indicating use of curcumin for the treatment of traumatic brain injury or Alzheimer's disease in future (Aiguo Wu, Zhe Ying, Femando Gomez-Pinilla. Dietary curcumin counteracts the outcome of traumatic brain injury on oxidative stress, synaptic plasticity, and cognition. Experimental Neurology, 2005, 1-9). Because of curcumin's pluripotency, oral safety, long history of use, and inexpensive cost, it's best suited for the prevention of multiple neurological conditions for which current therapeutics are less than optimal (Cole et al, A rationale for curcuminoids for the prevention or treatment of Alzheimer's disease. Curr Med Chem-Immun, Endoc, & Metab Agents. 2003; 3:15-25). Many neurodegenerative diseases involve the accumulation of protein aggregates, oxidative damage, and inflammation. β-secretase (BACE 1) a protease that cleavage few proteins that are particularly important in neural function, also cleavage β-amyloid (Aβ) peptide. When BACE1 is mutated or overactive it cut amyloid-beta precursor protein (APP) at a faster rate and even make specific cuts leading to subsequently γ-secretase beta amyloid is produced, along with the APP intracellular domain fragment and the sAPPβ fragment. Aβ may be cleaved into different lengths, the most common being 40 amino acids and 42 amino acids long, the latter one displaying less solubility. Aβ peptide is, thus, normally rapidly produced and equally rapidly degraded. However, at elevated concentrations, it has a strong tendency to self-aggregate to form Aβ plaques, poorly degradable, β-pleated sheet-rich oligomers, protofilaments, and, finally, filaments that have the histochemical staining properties of amyloid. It is also hypothesised that soluable part of Aβ which was cut from APP get attracted by metal ions and create toxicity at neural synapses. Aβ plaques and toxicity at neurological level is found in patients suffering from dementia, Alzheimer's disease or other pathological memory loss. Research shows curcumin may help the macrophages to clear the amyloid plaques, macrophages play an important role in the immune system, they help the body to fight against foreign proteins and then effectively clear them (Bowirrat Abdalla et al, Traditional and Modern Medicine Harmonizing the Two Approaches in the Treatment of Neurodegeneration (Alzheimer's Disease—Aβ) Chapter 10, http://dx.doi.org/10.5772/48558. 2012; 203). Curcumin has shown an ability to destabilize Aβ plaque formation, increase phagocytosis of Aβ, interact with metal ions and cholesterol, and reduce overall inflammation from escalatory pathology due to tau (Tau stabilize microtubules, but in its hyperphosphorylated state destabilizes microtubules, leading to inflammation within microglial cells and formation of neurofibrillary tangles, which trigger cell death).

Curcumin also shows the potential to inhibit cyclooxygenase-2 and NF-κB activity, reducing overall inflammation (Edward et al, Therapeutic Effects of Curcumin on Alzheimer's Disease; 2014; 145-152). Work in the field is promising despite the widely held belief that curcumin's poor systemic bioavailability precludes therapeutic utility outside of the colon. Nearly all the investigations on the pharmacological properties of turmeric have been focused on curcumin, anticonvulsant activities of curcumin has been demonstrated in several rodent models such as the iron-induced epileptogenesis, maximal electroshock, kainic acid-induced and pentylenetetrazole-kindling models. In addition to curcumin there are several other biologically active compounds in turmeric which have not received much attention, but lately have shown desirable pharmacological effects. The aromatic volatile oil part of turmeric also know as essential oil of turmeric (EOT) and the major compounds identified in turmeric volatile oil are ar-turmerone, turmerone, ar-curcumene, zingiberene, α-phellandrene, curlone, 1, 8-cineol and some other sesquiterpenes. According to the finding of G. Singh et al (2010). EOT includes aromatic (Ar)-turmerone (24.4%), alphaturmerone (20.5%) and beta-turmerone (11.1%) in fresh rhizome and aromatic-turmerone (21.4%), alpha-santalene (7.2%) and aromatic-curcumene (6.6%) in dry rhizome oil. Whereas, in oleoresins, the major components are alpha-turmerone (53.4%), beta-turmerone (18.1%) and aromatic-turmerone (6.2%) in fresh and aromatic-turmerone (9.6%), alpha-santalene (7.8%) and alpha-turmerone (6.5%) in dry rhizome. Alpha-turmerone, a major component in fresh rhizomes is only minor one in dry rhizomes (G. Singh et al. Comparative study of chemical composition and antioxidant activity of fresh and dry rhizomes of turmeric (Curcuma longa Linn). Food and Chemical Toxicology, Volume 48, Issue 4, April 2010, Pages 1026-1031). A study conducted on rats by Preeti et al. shows that Curcumin oil significantly reduces infarct size and improves neuro-score after ischemic insult induced by MCAo rats, they also showed iNOS-derived NO produced during ischemic injury was crucial for the up-regulation of ischemic injury targets and Curcumin oil down-regulates these targets this coincided with an increased survival rate of neurons. (Preeti Dohare, Puja Garg, Uma sharma, N R Jagannathan and Madhur Ray. Neuroprotective efficacy and therapeutic window of curcuma oil: in rat embolic stroke model, BMC Complementary and Alternative Medicine 2008, 8:55 doi: 10.1186/1472-6882-8-55). Aromatic turmerone, one of the major constituent of EoT has shown promising results in inducing proliferation of neural stem cells, which can help in treatment of neurological disease in future (Joerg Hucklenbroich, Rebecca Klein, Bernd Neumaier, Rudolf Graf, Gereon Rudolf Fink, Michael Schroeter and Maria Adele Rueger. Aromatic-turmerone induces neural stem cell proliferation in vitro and in vivo; Stem Cell Research & Therapy 2014 2014, 5:100).

However, while a few studies on the neuroprotective activity of turmeric oil have been performed but pharmacological effect of individual compounds has not yet identified (Dohare, P., et al Neuroprotective efficacy and therapeutic window of curcuma oil: in rat embolic stroke model. 55, 2008, BCM Complement Altern Med, Vol. 8. 17. Rathore, P., et al Curcuma oil: reduces early accumulation of oxidative product and is anti-apoptogenic in trasient focal ischemia in rat brain. 2008, Neurochem Res, Vol. 33, pp. 1672-82). Few exception in the field are the work of Antony et al (2005) to overcome the poor oral bioavailability of curcumin by blending curcuminoids with enriched essential oil of turmeric containing up to 45% Ar-turmerone (Antony et al. 2005; WO2006129323). Research work on non curcumin compounds of turmeric has been limited; it might me because of the limitation in the isolation of complex compounds from turmeric oil, especially from essential oil of turmeric.

There are other botanical components which incur similar physiological effect as turmeric compounds. The nature of action of different botanical compounds might differ for the same problem, and this feature can be exploited for enhanced activity. For instance there can be multiple plant extracts that act against beta secretase reduction, but the action might not be the same. The action might also vary according to the bioavailability of each plant extract. Like turmeric, Sesame is highly regarded for its use in traditional remedies. Sesame is a flowering plant in the genus Sesamum, found prevalently in Africa and some parts of India and China. Sesame indicum, is indigenous to India, it is a drought resistant species with high oil concentration in its seeds. Since ancient times consumption sesame seed oil is associated with youth, strength and rejuvenating property. Sesame oil contains a good amount of phenol, sesamin, sesamol and sesamolin and relatively small amounts of tocopherol which contributes to its superior oxidative stability.

Sesamin oil showed good antioxidant activity in comparison to canola oil. Sesame oil, in comparison to other dietary oils such as ground nut and sunflower, offers better protection against increased blood pressure, hyperlipidemia and lipid peroxidation by increasing enzymatic and non-enzymatic antioxidants (Sankar et al., 2005. Modulation of blood pressure, lipid profiles and redox status in hypertensive patients taking different edible oils. Clinica Chimica Acta 355, 97-104). Fu-Chou Cheng et al. found out from their study that oral administration of sesamin or the crude sesame oil extract is neuroprotective in terms of reducing ischemic damage in experimental MCA+CCA occluded gerbils (Fu-Chou Chengl, Tzyy-Rong Jinn. Rolis C. W. Hou, Jason T. C. Tzen. Neuroprotective Effects of Sesamin and Sesamolin on Gerbil Brain in Cerebral Ischemia. International Journal of Biomedical Science, 2006, 284-288). Sesame seeds have positive effect on pathological memory related problem such as Alzheimer's disease, Shigeru Katayama et al suggest that long term consumption of sesamolin, a liganin found in sesame oil might helps in inhibiting accumulation of pathogenic β-amyloid (Aβ) in the brain (Shigeru Katayama. Haruka Sugiyama, Shoko Kushimoto, Yusuke Uchiyama, Masato Hirano, and Soichiro Nakamura. Effects of Sesaminol Feeding on Brain AP Accumulation in a Senescence-Accelerated Mouse-Prone 8; Department of Bioscience and Biotechnology, Shinshu University. 8304 Minamiminowa, Kamiina, Nagano 399-4598, Japan, Agric. Food Chem., 2016, 64 (24), pp 4908-4913).

Methanol extract of sesame seed have shown promising antioxidative activity, further analysis also found out the main constituents to be lignan glycosides and some unknown brown materials. (Shyu, Y. S.; Hwang, L. S. Antioxidant activity of the crude extract of lignan glycosides from unroasted Burma black sesame meal. Food Res. Int. 2002, 35, 357-365). A study conducted by Fu-Chou Cheng et al (2006), gerbils were repeatedly administered with sesamin or a crude sesame oil extract containing both sesamin and sesamolin prior to a focal cerebral ischemia. Oral administration of sesamin or the crude sesame oil extract was found to be neuroprotective in terms of reducing ischemic damage in experimental MCA+CCA occluded gerbils (Fu-Chou Cheng et al, Neuroprotective Effects of Sesamin and Sesamolin on Gerbil Brain in Cerebral Ischemia. International journal of Biomedical science, 2006. 284-288).

Analysis of different samples of sesame seeds indicated that the content of sesaminol triglucoside ranged from 36 to 1560 mg/100 g of seed (mean 637±312) and that of sesaminol diglucoside ranged from 0 to 493 mg/100 g of seed (mean 75±95) (Ali A. Moazzami, Rolf E. Andersson, and Afaf Kamal-Eldin, HPLC Analysis of Sesaminol Glucosides in Sesame Seeds. J. Agric. Food Chem., 2006, 54 (3), pp 633-638). Sesamin and sesamolin are part of the triglucoside and are extractable in oil, and sesame seed and the unrefined oil thereof contain these two lignans at a ratio of about 2:1. The sesamin preparation obtained as a by-product of the refining of edible sesame oil therefore consists of a 1:1 ratio of sesamin and episesamin, and has been tested extensively for physiological activity in animals. (Jin Seon Lim, Yoshikazu Adachi, Yoko Takahashi and Takashi Ide, Comparative analysis of sesame lignans (sesamin and sesamolin) in affecting hepatic fatty acid metabolism in rats; British Journal of Nutrition (2007), 97, 85-95). Enrichment of Sesame oil for nutraceutical applications by M. V. Reshma et al has managed to produce a 46:54 sesamin to sesamolin extract with 94.36% purity of liganin from a 84:16 sesmin oil cristal (M. V. Reshma. C. Balachandran, C. Arumughan, A. Sunderasan, Divya Sukumaran, Shiny Thomas, S. S. Saritha; Extraction, separation and characterisation of sesame oil lignin for nutraceutical applications; Food Chemistry 120 (2010) 1041-1046).

There are multiple studies to show the beneficial effect of plant extracts on various health related adversities. But synergetic effect of different plant extracts on mammalian health is yet to be explored. Some embodiments of the disclosure are directed towards a composition comprising different plant extracts, to achieve a higher efficacy than their natural counterparts.

### SUMMARY

Disclosure relates to a medicinal composition/formulation to modulate the activity of beta secretase.

The disclosure provides a composition for enhancing the curcumin, α-turmerone and sesamolin availability in brain and the composition comprises of curcuminoid, essential oil of turmeric with α-turmerone, and sesame seed extract enriched with sesamolin.

Compositions for modulating beta secretase activity are provided. The composition includes a curcuminoid mixture and an essential oil of turmeric. The curcuminoid mixture includes curcumin, demethoxycurcumin. and, bisdemethoxycurcumin. The essential oil of turmeric includes about 30% to about 80% of α-turmerone. A weight ratio of the curcuminoid mixture to the essential oil of turmeric ranges from about 1:50 to about 99:1. The composition also enhances bioavailability of a constituent such as curcumin, demethoxycurcumin, bisdemethoxycurcumin, α-turmerone, β-turmerone, and, ar-turmerone. A method of enhancing bioavailability of a constituent in brain, plasma or serum is provided. The constituent can be curcumin, demethoxycurcumin, bisdemethoxycurcumin, α-turmerone, β-turmerone, or, ar-turmerone. A method of preparing the composition is provided. The method includes suspending a powdered extract of curcuminoid mixture in water to form a suspension. Then essential oil of turmeric is added to the suspension to obtain a mixture. The mixture is pulverized to obtain a slurry. Water is stripped from the slurry to obtain a powder of the composition having curcuminoid mixture and essential oil of turmeric having about 30% to about 80% α-turmerone. The powdered extract curcuminoid mixture is prepared by drying and powdering turmeric rhizomes to form a powdered rhizome. Then extracting the powdered rhizome with ethyl acetate at 78° C. to obtain a residue and a supernatant. Then cooling the supernatant at 4° C. to obtain crystals and a liquid. Then powdering the crystals to obtain powdered extract of curcuminoid mixture, wherein the curcuminoid mixture consists of curcumin, demethoxycurcumin, and, bisdemethoxycurcumin. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30% α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 60% α-turmerone is further purified by silica chromatography column using 99%, hexane elution followed by elution with 1% chloroform to obtain the essential oil of turmeric having about 80% α-turmerone.

Compositions for inhibiting beta secretase activity are provided, wherein the composition has a curcuminoid mixture and a combination. The combination includes an essential oil of turmeric and an extract of sesame oil. The curcuminoid mixture includes curcumin, demethoxycurcumin, and, bisdemethoxycurcumin. The essential oil of turmeric includes about 30% to about 80% of α-turmerone. The extract of sesame oil includes about 30% to about 80% sesamolin. A weight ratio of the curcuminoid mixture to the combination is about 70:30. A weight ratio of the essential oil of turmeric to the extract of sesame oil ranges from about 1:3 to about 8:1. The composition also has the property of enhancing bioavailability of a constituent. The constituent is curcumin, demethoxycurcumin, bisdemethoxycurcumin, α-turmerone, β-turmerone, ar-turmerone, or, sesamolin. Method for enhancing bioavailability of a constituent in brain, serum and/or plasma by administering the compositions is provided. The constituent includes curcumin, demethoxycurcumin, bisdemethoxycurcumin, α-turmerone, β-turmerone, ar-turmerone, or, sesamolin. A method of preparing the composition is provided. The method includes suspending a powdered extract of curcuminoid mixture in water to form a suspension. Followed by adding a combination to the suspension to obtain a mixture. Then pulverizing the mixture to obtain a slurry, and stripping water from the slurry to obtain a powder of the composition. The combination includes an essential oil of turmeric, and, an extract of sesame oil. The powdered extract curcuminoid mixture is prepared by drying and powdering turmeric rhizomes to form a powdered rhizome. Then extracting the powdered rhizome with ethyl acetate at 78° C. to obtain a residue and a supernatant. Then cooling the supernatant at 4° C. to obtain crystals and a liquid. Then powdering the crystals to obtain powdered extract of curcuminoid mixture, wherein the curcuminoid mixture consists of curcumin, demethoxycurcumin, and, bisdemethoxycurcumin. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30% α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 600/α-turmerone is further purified by silica chromatography column using 99% hexane elution followed by elution with 1% chloroform to obtain the essential oil of turmeric having about 80% α-turmerone. Methods of preparing various extracts of sesame oil are provided. A first extract of sesame oil from sesame seed is prepared. Next, n-hexane is added to the first extract of sesame oil to obtain a mixture. The mixture is passed through an adsorption column and obtaining a first hexane elute and a first ethyl acetate elute. Then evaporating the first ethyl acetate elute to obtain a second extract of sesame oil. Next, allowing the second extract of sesame oil to set overnight to form a precipitate. Then, filtering the precipitate, wherein the precipitate includes crystals having about 20% sesamolin. Followed by loading the crystals having about 20% sesamolin on a silica chromatography column. Then eluting the silica chromatography column with 85% hexane in ethyl acetate to obtain a second hexane elute and a second ethyl acetate elute. The second ethyl acetate includes about 40% sesamolin. Next, loading the second ethyl acetate elute onto a silica chromatography column and eluting (1) first with 91% hexane to obtain a 91% hexane elute, followed by a second elution with 9% ethyl acetate to obtain a 9% ethyl acetate elute which is a third extract of sesame oil having about 60% sesamolin. Then loading the third extract of sesame oil onto a silica chromatography column and eluting with 93% hexane and 7% ethyl acetate to obtain a 93% hexane elute and a 7% ethyl acetate elute. The 7% ethyl acetate elute is a fourth extract of sesame oil having about 80% sesamolin. Alternately, the second ethyl acetate elute having about 40% sesamolin is loaded onto a silica chromatography column and eluted with a gradient of 92% hexane and 8% ethyl acetate to obtain a 92% hexane elute and an 8% ethyl acetate elute. The 8% ethyl acetate elute is a fifth extract of sesame oil having about 55% sesamolin. The extracts of sesame oil can have about 20% sesamolin, about 40% sesamolin, about 55% sesamolin, about 60% sesamolin, about 80% sesamolin, and, combinations thereof to obtain other sesame oil extracts.

Compositions for inhibiting beta secretase activity are provided. The composition is a combination of an essential oil of turmeric and an extract of sesame oil. In some embodiments the essential oil of turmeric includes about 30% to about 80% of α-turmerone. The extract of sesame oil includes about 30% to about 80% sesamolin. The weight ratio of the essential oil of turmeric to the extract of sesame oil ranges from about 1:3 to about 8:1. The compositions enhance bioavailability of a constituent selected from the group consisting of α-turmerone, β-turmerone, ar-turmerone, or, sesamolin. Methods for enhancing the bioavailability of a constituent in brain, serum or plasma by administering the composition are provided. The constituent can be α-turmerone. 0-turmerone, ar-turmerone, or, sesamolin. Methods of preparing the compositions are provided. Methods include mixing essential oil of turmeric and extract of sesame oil to obtain a first mixture. Then warming the first mixture to 65° C. to obtain a warm mixture. Next, blending the warm mixture to obtain a blend of the composition. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30%, α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 60% α-turmerone is further purified by silica chromatography column using 99% hexane elution followed by elution with 1% chloroform to obtain the essential oil of turmeric having about 80% α-turmerone. The extract of sesame oil is prepared by extracting a first extract of sesame oil from sesame seed. Then adding n-hexane to the first extract of sesame oil to obtain a mixture. Next, passing the mixture through an adsorption column and obtaining a first hexane elute and a first ethyl acetate elute. Then evaporating the first ethyl acetate elute to obtain a second extract of sesame oil. Next, allowing the second extract of sesame oil to set overnight to form a precipitate. Followed by filtering the precipitate which includes crystals having about 20% sesamolin.

Further, the method includes loading the crystals having about 20% sesamolin on a silica chromatography column. Then eluting the silica chromatography column with 85% hexane in ethyl acetate to obtain a second hexane elute and a second ethyl acetate elute. The second ethyl acetate extract includes about 40% sesamolin. Then loading the second ethyl acetate elute onto a silica chromatography column and eluting first with 91% hexane to obtain a 91% hexane elute. Next, eluting with 9% ethyl acetate to obtain a 9% ethyl acetate elute which provides a third extract of sesame oil having about 60% sesamolin. The method further includes loading the third extract of sesame oil onto a silica chromatography column and eluting first with 93% hexane and then 7% ethyl acetate to obtain a 93% hexane elute and a 7% ethyl acetate elute. The 7% ethyl acetate elute includes a fourth extract of sesame oil having about 80% sesamolin. The method includes loading the crystals having about 40% sesamolin on a silica chromatography column and eluting with a gradient of 92% hexane and 8% ethyl acetate to obtain a 92% hexane elute and an 8% ethyl acetate elute. The 8% ethyl acetate elute is a fifth extract of sesame oil having about 55% sesamolin.

Dosage form of the compositions such as capsule, tablet, granule, sachet, powder, paste, infusion, ampoule, solution, suspension, emulsion, pills, or, cream are provided. Oral dosage forms of the compositions are provided. Compositions to inhibit beta secretase activity in brain. Methods for inhibiting beta secretase in brain administering the compositions are provided. A method for treating a central nervous system disorder by administering the compositions is provided. The central nervous system disorders treated include epilepsy, migraine, Huntington's disease. Alzheimer's disease, depression, Parkinson's disease, Tourette syndrome, dystonia, multiple sclerosis, meningitis, lupus, fibromyalgia, or, bipolar disorder.

### BRIEF DESCRIPTION OF THE DRAWINGS

The above objectives and advantages of the disclosed teachings will become more apparent by describing in detail preferred embodiments thereof with reference to the attached drawings in which:

FIG. 1 provides method of preparation of regular turmeric extract.

FIG. 2 provides method of preparation of Essential oil of turmeric enriched with α-turmerone.

FIG. 3 provides method of preparation of combination of curcuminoids and essential oil of turmeric with 80% α-turmerone in 12:1 ratio.

FIG. 4 provides method of preparation of combination of curcuminoids and essential oil of turmeric with 60% α-turmerone in 12:1 ratio.

FIG. 5 provides method of preparation of combination of curcuminoids and essential oil of turmeric with 40% α-turmerone in 12:1 ratio.

FIG. 6 provides method of preparation of combination of curcuminoids and essential oil of turmeric with 30% α-turmerone in 12:1 ratio.

FIG. 7 provides method of preparation of combination of curcuminoids and essential oil of turmeric with 30% α-turmerone in 10:1 ratio.

FIG. 8 provides method of preparation of Essential oil of turmeric with 45% Ar-turmerone.

FIG. 9 provides method of preparation of combination of curcuminoids and essential oil of turmeric with 45% Ar-turmerone in 12:1 ratio.

FIG. 10 provides method of preparation of extract of sesame seed with different ratios of Sesamin and Sesamolin.

FIG. 11 provides method of preparation of combination of essential oil of turmeric enriched with α-turmerone (30%) and sesamolin (20%) in 8:1 ratio.

FIG. 12 provides method of preparation of combination of essential oil of turmeric enriched with α-turmerone (30%) and sesamolin (40%) in 8:1 ratio.

FIG. 13 provides method of preparation of combination of essential oil of turmeric enriched with α-turmerone (60%) and sesamolin (40%) in 8:1 ratio.

FIG. 14 provides method of preparation of combination of essential oil of turmeric enriched with α-turmerone (30%) and sesamolin (40%) in 5:1 ratio.

FIG. 15 provides method of preparation of combination of essential oil of turmeric enriched with α-turmerone (30%) and sesamolin 80% in 8:1 ratio.

FIG. 16 provides method of preparation of combination of curcuminoid and 8:1 combination of essential oil of turmeric enriched with 30% α-turmerone and 40% sesamolin in 70:30 ratio.

### DETAILED DESCRIPTION

Central nervous system disorders are a group of diseases and conditions that affect the health and functioning of the spinal cord and brain. There are many different types of central nervous system disorders, some of which include epilepsy, migraine, Huntington's disease, Alzheimer's disease, Parkinson's disease, Tourette syndrome, dystonia, multiple sclerosis, meningitis, lupus, fibromyalgia, and bipolar disorder. While central nervous system disorders can vary greatly from each other, all the disorders cause a loss of sufficient, intact nervous system circuits that orchestrate particular functions. Neurodegenerative diseases are incurable and debilitating conditions that result in progressive degeneration and/or death of nerve cells. This causes problems with movement (called ataxias), or mental functioning (called dementias). Protein aggregation is a common feature of many neurodegenerative diseases, and it is assumed that the aggregation process plays a central role in pathogenesis. Alzheimers disease (AD) is a devastating neurodegenerative disorder of the brain characterized by accumulation and deposition of amyloid β (Aβ) peptide. AD affects about 5.4 million people in the United States alone, and that number is projected to reach 12-16 million by the year 2050, as the elderly worldwide is increasing, especially in China and India, by 2030 it will be the highest in human history. The main causative factor of AD is senile plaque produced by the amyloid β peptide, which itself is produced by degradation of the amyloid precursor protein catalyzed by secretases. Among the 3 subtypes of secretases, β-secretase is recognized as a promising target for the prevention and treatment of AD since inhibition of the enzyme is expected to possess fewer side effects compared to other secretases.

Among treatments for AD, there is a renewed interest in curcumin as a potential medication. Curcuminoids from turmeric (rhizome) have been reported to possess β-secretase inhibitory activities. During our research work to enhancing the bioavailability of curcuminoid, we discovered enhanced activity by compounds from essential oil of turmeric, especially from alpha-turmerone enriched essential oil of turmeric. In our studies we found that, α-turmerones the least polar compounds from turmeric, showed significant potent β-secretase inhibitory activity.

Sesame seed is a popular food or spice as well as a source of oil. Sesame oil is known to be a rich source of unsaturated fatty acids including linoleic acid and olenic acid. Extract prepared from Sesamum indicum seeds showed potent f-secretase inhibitory activity. The active principles were determined to be sesamin and sesamolin, typical lignans in S. indicum.

Sesame seed extracts are tested for activity towards β-secretase, the results are unexpected and encouraging. The purified sesamolin has inhibitory activity towards f-secretase, especially in brain; this implies that sesamolin can be used for treatment of Alzheimer's disease.

In one embodiment, essential oil of turmeric enriched with α-turmerone blended with curcuminoids had shown promising inhibitory activity towards β-secretase in brain. This was a new finding and the efficacy was unexpected.

In another embodiment it was found that a formulation comprising turmeric extracts and sesame seed extract showed enhanced bioavailability of sesame extract, in particular in brain and an unexpected potency towards inhabiting β-secretase was observed. A synergy among the turmeric and sesame actives has led to the enhanced potency of the formulation. In vitro study has shown great potential for sesamolin as an inhibitor of β-secretase and as a treatment for Alzheimer's disease.

Some embodiments provide a formulation to modulate the activity of beta secretase containing curcuminoid and essential oil of turmeric with α-turmerone, the formulation further containing sesame seed extract with more than 35% sesamolin.

The disclosure relates to a formulation to modulate the activity of beta secretase, formulation containing curcuminoid and purified essential oil of turmeric enriched with α-turmerone. A composition for enhancing the curcumin and α-turmerone availability in brain containing curcuminoid and purified essential oil of turmeric wherein curcumin is the main constituent of curcuminoid and wherein α-turmerone is the main constituent of the essential oil of turmeric. A composition for enhancing the curcuminoid and α-turmerone availability in brain is containing curcuminoid and purified essential oil of turmeric. In some embodiments, the weight ratio of the curcuminoid mixture to the added purified essential oil of turmeric enriched with α-turmerone ranges from about 1:50 to about 99:1. According to another embodiment, the purified essential oil of turmeric contains α-turmerone with 80% purity. The curcuminoid mixture includes curcumin, demethoxycurcumin and bisdemethoxycurcumin. The purified essential oil of turmeric includes α-turmerone, β-turmerone and Ar-turmerone. Compositions for modulating beta secretase activity are provided. The composition includes a curcuminoid mixture and an essential oil of turmeric. The curcuminoid mixture includes curcumin, demethoxycurcumin. and, bisdemethoxycurcumin. The essential oil of turmeric includes about 30% to about 80% of α-turmerone. A weight ratio of the curcuminoid mixture to the essential oil of turmeric ranges from about 1:50 to about 99:1. The composition also enhances bioavailability of a constituent such as curcumin, demethoxycurcumin, bisdemethoxycurcumin. α-turmerone, β-turmerone, and, ar-turmerone. A method of enhancing bioavailability of a constituent in brain, plasma or serum is provided. The constituent can be curcumin, demethoxycurcumin, bisdemethoxycurcumin, α-turmerone, β-turmerone, or, ar-turmerone.

In some embodiments, the weight ratio of the curcuminoid to the essential oil of turmeric with α-turmerone ranges from about 1:1 to about 90:1. In some embodiments, the weight ratio of the curcuminoid to the essential oil of turmeric with α-turmerone ranges from about 1:1 to about 3:1. The weight ratio of the curcuminoid to the essential oil of turmeric with α-turmerone can be varied from about 3:1 to about 99:1. In some embodiments, the weight ratio of the curcuminoid to the essential oil of turmeric with α-turmerone ranges from about 1:1 to about 70:1. In some embodiments, the weight ratio of the curcuminoid to the essential oil of turmeric with α-turmerone ranges from about 1:1 to about 45:1. In some embodiments, the weight ratio of the curcuminoid to the essential oil of turmeric with α-turmerone ranges from about 3:1 to about 50:1. In some embodiments, the weight ratio of the curcuminoid to the essential oil of turmeric with α-turmerone ranges from about 8:1 to about 25:1. In some embodiments, the weight ratio of the curcuminoid to the essential oil of turmeric with α-turmerone is about 90:7. In some embodiments, the weight ratio of the curcuminoid to the essential oil of turmeric with α-turmerone is about 90:8. In some embodiments, the weight ratio of the curcuminoid to the essential oil of turmeric with α-turmerone is about 90:9. In some embodiments, the weight ratio of the curcuminoid to the essential oil of turmeric with α-turmerone is about 89:9. In some embodiments, the weight ratio of the curcuminoid to the essential oil of turmeric with α-turmerone is about 89:8. In one embodiment, the ratio is about 85:15. In another embodiment, the ratio is about 92:8. In another embodiment, the ratio is about 95:5. In another embodiment the weight ratio is about 10:1. In some embodiments, the weight ratio is about 12:1. In some embodiments, the weight ratio of the curcuminoid to the essential oil of turmeric with α-turmerone is about 1:2. In some embodiments, the weight ratio of the curcuminoid to the essential oil of turmeric with α-turmerone is about 2:1. In some embodiments, the weight ratio of the curcuminoid to the essential oil of turmeric with α-turmerone ranges from about 1:50 to about 99:1.

Different ratios of curcuminoid and essential oil of turmeric enriched with α-turmerone which shows enhanced bioavailability of curcumin, demethoxycurcumin and bisdemethoxycurcumin and β-secretase inhibitory activity are as following, but not limiting to;

| Ratios of Curcuminoid:EOT | | |
|---|---|---|
| with 80% α-turmerone | curcuminoids (%) | EOT (%) |
| 99:1 | 94.05 | 1 |
| 95:5 or 19:1 | 90.25 | 5 |
| 90:4 or 22.5:1 | 90.95 | 4.25 |
| 90:5 or 18:1 | 90 | 5.26 |
| 90:6 or 15:1 | 89.06 | 6.25 |
| 90:7 or 12.8:1 | 88.14 | 7.21 |
| 90:8 or 11.3:1 | 87.24 | 8.16 |
| 90:9 or 10:1 | 86.36 | 9.09 |
| 90:10 or 9:1 | 85.5 | 10 |
| 90:20 or 4.5:1 | 77.72 | 18.18 |
| 89:9 or 9.8:1 | 86.27 | 9.18 |
| 80:9 or 8.8:1 | 85.39 | 10.11 |
| 80:20 or 4:1 | 76 | 20 |
| 70:20 or 3.5:1 | 73.88 | 22.22 |
| 70:1 | 93.99 | 1.40 |
| 60:1 | 93.44 | 1.63 |
| 50:1 | 93.13 | 1.96 |
| 40:1 | 92.68 | 2.43 |
| 75:25 or 3:1 | 71.25 | 25 |
| 1:1 | 47.5 | 50 |
| 1:2 | 31.67 | 66.66 |
| 1:5 | 15.83 | 83.33 |
| 1:10 | 8.63 | 90.90 |
| 1:20 | 4.5 | 95.23 |
| 1:50 | 1.86 | 98.03 |

In some embodiments of the composition, curcuminoid and added essential oil of turmeric enriched with α-turmerone, the curcuminoid ranges, by weight, from about 24% to about 96%. In some embodiments of the composition comprising curcuminoid and added essential oil of turmeric, the curcuminoid ranges, by weight, from about 30% to about 96%. In some embodiments of the composition of curcuminoid and added essential oil of turmeric with α-turmerone, the curcuminoid ranges, by weight, from about 40% to about 75%. In some embodiments the composition comprising curcuminoid and added essential oil of turmeric with α-turmerone, the curcuminoid ranges, by weight, from about 50% to about 60%. In some embodiments of the composition of curcuminoid and essential oil of turmeric with α-turmerone, the curcumin ranges, by weight, from about 25% to about 73%. In some embodiments of the composition of curcuminoid and essential oil of turmeric with α-turmerone, the curcumin ranges, by weight, from about 40% to about 70%. In some embodiments the composition of curcuminoid and added essential oil of turmeric with α-turmerone, the curcumin ranges, by weight, from about 60% to about 73%.

In some embodiments of the composition of curcuminoid and added essential oil of turmeric with α-turmerone, the demethoxycurcumin ranges, by weight, from about 10% to about 50%. In some embodiments of the composition of curcuminoid and added essential oil of turmeric with α-turmerone, the demethoxycurcumin ranges, by weight, from about 10% to about 25%. In some embodiments of the composition of curcuminoid and added essential oil of turmeric with α-turmerone, the bisdemethoxycurcumin ranges, by weight, from about 2% to about 50%. In some embodiments of the composition of curcuminoid and added essential oil of turmeric with α-turmerone, the bisdemethoxycurcumin ranges, by weight, from about 2% to about 25%.

In some embodiments of the composition of curcuminoid and added essential oil of turmeric with α-turmerone, the purity of α-turmerone ranges from about 10% to about 80%. In some embodiments of the composition of curcuminoid and added essential oil of turmeric with α-turmerone, the purity of α-turmerone ranges from about 30% to about 80%. In some embodiments of the composition of curcuminoid and added essential oil of turmeric with α-turmerone, the purity of α-turmerone ranges from about 60% to about 80%.

Some embodiments provide a composition for enhancing the curcumin and α-turmerone availability in brain containing curcuminoid and essential oil of turmeric with α-turmerone, the composition further includes sesamolin and sesamin, the β-secretase inhibitory compound from sesame seed extract with more than 35% sesamolin. In some embodiment of the composition the weight ratio of essential oil of turmeric with α-turmerone to sesamin-sesamolin is 8:1. In some embodiment of the composition the weight ratio of essential oil of turmeric with α-turmerone to sesamin-sesamolin is 5:1. In some embodiment of the composition the weight ratio of essential oil of turmeric with α-turmerone to sesamin-sesamolin is 3:1.

Compositions for inhibiting beta secretase activity are provided, wherein the composition has a curcuminoid mixture and a combination. The combination includes an essential oil of turmeric and an extract of sesame oil. The curcuminoid mixture includes curcumin, demethoxycurcumin, and, bisdemethoxycurcumin. The essential oil of turmeric includes about 30% to about 80% of α-turmerone. The extract of sesame oil includes about 30% to about 80% sesamolin. A weight ratio of the curcuminoid mixture to the combination is about 70:30. A weight ratio of the essential oil of turmeric to the extract of sesame oil ranges from about 1:3 to about 8:1. The composition also has the property of enhancing bioavailability of a constituent. The constituent is curcumin, demethoxycurcumin, bisdemethoxycurcumin, α-turmerone, β-turmerone, ar-turmerone, or, sesamolin.

Some embodiments provide a composition for enhancing the curcumin and α-turmerone availability in brain containing curcuminoid and essential oil of turmeric with α-turmerone, the composition further includes sesamin and sesamolin in a ratio of about 2:1 to 1:90, wherein sesamolin content in the extract is above 35%. In some embodiment of the composition the weight ratio of essential oil of turmeric with α-turmerone to sesamin-sesamolin is 1:1. In some embodiment of the composition the weight ratio of essential oil of turmeric with α-turmerone to sesamin-sesamolin is 1:2. In some embodiment of the composition the weight ratio of essential oil of turmeric with α-turmerone to sesamin-sesamolin is 1:3.

Some embodiments provide a composition for enhancing the availability of sesamolin in brain, formulation containing curcuminoid and essential oil of turmeric with α-turmerone, the composition further includes sesamolin and sesamin, the β-secretase inhibitory compound from sesame seed extract with more than 35% sesamolin. In some embodiment of the composition the weight ratio of essential oil of turmeric enriched with α-turmerone to sesamin-sesamolin is 8:1.

In some embodiments of the composition the weight ratio of curcuminoid and combination of essential oil of turmeric enriched with α-turmerone and sesamolin is 70:30.

Some embodiments provide a process of extraction of curcuminoid from turmeric. Some embodiment provides process of extraction of essential oil of turmeric and enrichment of α-turmerone to a purity of more than 10%.

Compositions for inhibiting beta secretase activity are provided. The composition is a combination of an essential oil of turmeric and an extract of sesame oil. In some embodiments the essential oil of turmeric includes about 30% to about 80% of α-turmerone. The extract of sesame oil includes about 30% to about 80% sesamolin. The weight ratio of the essential oil of turmeric to the extract of sesame oil ranges from about 1:3 to about 8:1. The compositions enhance bioavailability of a constituent selected from the group consisting of α-turmerone, β-turmerone, ar-turmerone, or, sesamolin.

A method of extraction of curcuminoids includes treating rhizomes of turmeric with a solvent, followed by solvent stripping, and steam distilling to obtain an essential-oil free extract. Suitable solvents for curcuminoid extraction include ethanol, acetone, hexane, ethyl acetate, dicholoroethane, chloroform, etc. The extraction is conveniently carried out at moderate temperatures (40-55° C.) and the solvent is partially removed to yield a concentrate containing 30-60% solids. The extract free from essential oil is cooled to about 4° C. to allow the curcuminoids to crystallize. The curcuminoids are then separated by filtration, centrifugation or any other method of solid-liquid separation well-known in the art. In some embodiments, 95% of the separated crystals are composed of curcumin. demethoxycurcumin and bisdemethoxycurcumin.

Methods of preparing compositions are provided. The method includes suspending a powdered extract of curcuminoid mixture in water to form a suspension. Then essential oil of turmeric is added to the suspension to obtain a mixture. The mixture is pulverized to obtain a slurry. Water is stripped from the slurry to obtain a powder of the composition having curcuminoid mixture and essential oil of turmeric having about 30% to about 80% α-turmerone. The powdered extract curcuminoid mixture is prepared by drying and powdering turmeric rhizomes to form a powdered rhizome. Then extracting the powdered rhizome with ethyl acetate at 78° C. to obtain a residue and a supernatant. Then cooling the supernatant at 4° C. to obtain crystals and a liquid. Then powdering the crystals to obtain powdered extract of curcuminoid mixture, wherein the curcuminoid mixture consists of curcumin, demethoxycurcumin, and, bisdemethoxycurcumin. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30% α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 60% α-turmerone is further purified by silica chromatography column using 99% hexane elution followed by elution with 1% chloroform to obtain the essential oil of turmeric having about 80% α-turmerone.

In some embodiments, the method of preparing the composition includes suspending a powdered extract of curcuminoid mixture in water to form a suspension. Followed by adding a combination to the suspension to obtain a mixture. Then pulverizing the mixture to obtain a slurry, and stripping water from the slurry to obtain a powder of the composition. The combination includes an essential oil of turmeric, and, an extract of sesame oil. The powdered extract curcuminoid mixture is prepared by drying and powdering turmeric rhizomes to form a powdered rhizome. Then extracting the powdered rhizome with ethyl acetate at 78° C. to obtain a residue and a supernatant. Then cooling the supernatant at 4° C. to obtain crystals and a liquid. Then powdering the crystals to obtain powdered extract of curcuminoid mixture, wherein the curcuminoid mixture consists of curcumin, demethoxycurcumin, and, bisdemethoxycurcumin. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30% α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 60% α-turmerone is further purified by silica chromatography column using 99% hexane elution followed by elution with 1% chloroform to obtain the essential oil of turmeric having about 80% α-turmerone. Methods of preparing various extracts of sesame oil are provided. A first extract of sesame oil from sesame seed is prepared. Next, n-hexane is added to the first extract of sesame oil to obtain a mixture. The mixture is passed through an adsorption column and obtaining a first hexane elute and a first ethyl acetate elute. Then evaporating the first ethyl acetate elute to obtain a second extract of sesame oil, Next, allowing the second extract of sesame oil to set overnight to form a precipitate. Then, filtering the precipitate, wherein the precipitate includes crystals having about 20% sesamolin. Followed by loading the crystals having about 20% sesamolin on a silica chromatography column. Then eluting the silica chromatography column with 85% hexane in ethyl acetate to obtain a second hexane elute and a second ethyl acetate elute. Next, loading the second ethyl acetate elute onto a silica chromatography column and eluting (1) first with 91% hexane to obtain a 91% hexane elute, followed by a second elution with 9% ethyl acetate to obtain a 9% ethyl acetate elute which is a third extract of sesame oil having about 60% sesamolin. Then loading the third extract of sesame oil onto a silica chromatography column and eluting with 93% hexane and 7% ethyl acetate to obtain a 93% hexane elute and a 7% ethyl acetate elute. The 7% ethyl acetate elute is a fourth extract of sesame oil having about 80% sesamolin. Alternately, the second ethyl acetate elute is loaded onto a silica chromatography column and eluted with a gradient of 92% hexane and 8% ethyl acetate to obtain a 92% hexane elute and an 8% ethyl acetate elute. The 8% ethyl acetate elute is a fifth extract of sesame oil having about 55% sesamolin. The extracts of sesame oil can have about 20% sesamolin, about 40% sesamolin, about 55% sesamolin, about 60% sesamolin, about 80% sesamolin, and, combinations thereof to obtain other sesame oil extracts.

In some embodiments, the methods include mixing essential oil of turmeric and extract of sesame oil to obtain a first mixture. Then warming the first mixture to 65° C. to obtain a warm mixture. Next, blending the warm mixture to obtain a blend of the composition. The essential oil of turmeric is prepared by flaking fresh rhizome of turmeric to obtain flaked turmeric rhizomes. Then distilling the flaked turmeric rhizome in water at 64° C. to obtain distilled flakes of turmeric. Then soaking the distilled flakes of turmeric in water to obtain soaked flakes. Then heating the soaked flakes of turmeric to 97° C. to obtain the essential oil of turmeric having about 30% α-turmerone. Next, purifying the essential oil of turmeric having about 30% α-turmerone by silica chromatography column eluting first with 99% hexane elution followed by eluting with 1% ethyl acetate to obtain the essential oil of turmeric having about 40% α-turmerone. The essential oil of turmeric having 30% α-turmerone is further purified by silica chromatography column using 98.5% hexane elution followed by elution with 1.5% ethyl acetate to obtain the essential oil of turmeric having about 60% α-turmerone. The essential oil of turmeric having about 60% α-turmerone is further purified by silica chromatography column using 99% hexane elution followed by elution with 1% chloroform to obtain the essential oil of turmeric having about 80% α-turmerone. The extract of sesame oil is prepared by extracting a first extract of sesame oil from sesame seed. Then adding n-hexane to the first extract of sesame oil to obtain a mixture. Next, passing the mixture through an adsorption column and obtaining a first hexane elute and a first ethyl acetate elute. Then evaporating the first ethyl acetate elute to obtain a second extract of sesame oil. Next, allowing the second extract of sesame oil to set overnight to form a precipitate. Followed by filtering the precipitate which includes crystals having about 20% sesamolin. Further, the method includes loading the crystals having about 20% sesamolin on a silica chromatography column. Then eluting the silica chromatography column with 85% hexane in ethyl acetate to obtain a second hexane elute and a second ethyl acetate elute. Then loading the second ethyl acetate elute onto a silica chromatography column and eluting first with 91% hexane to obtain a 91% hexane elute. Next, eluting with 9% ethyl acetate to obtain a 9% ethyl acetate elute which provides a third extract of sesame oil having about 60% sesamolin. The method further includes loading the third extract of sesame oil onto a silica chromatography column and eluting first with 93% hexane and then 7% ethyl acetate to obtain a 93% hexane elute and a 7% ethyl acetate elute. The 7% ethyl acetate elute includes a fourth extract of sesame oil having about 80% sesamolin. The method includes loading the crystals having about 40% sesamolin on a silica chromatography column and eluting with a gradient of 92% hexane and 8% ethyl acetate to obtain a 92% hexane elute and an 8% ethyl acetate elute. The 8% ethyl acetate elute is a fifth extract of sesame oil having about 55% sesamolin.

Dosage form of the compositions such as capsule, tablet, granule, sachet, powder, paste, infusion, ampoule, solution, suspension, emulsion, pills, or, cream are disclosed. Oral dosage forms of the compositions are disclosed. Compositions to inhibit beta secretase activity in brain are disclosed. Methods for inhibiting beta secretase in brain administering the compositions are provided. Methods for treating a central nervous system disorder by administering the compositions are disclosed. The central nervous system disorders treated include epilepsy, migraine, Huntington's disease, Alzheimer's disease, depression, Parkinson's disease, Tourette syndrome, dystonia, multiple sclerosis, meningitis, lupus, fibromyalgia, or, bipolar disorder.

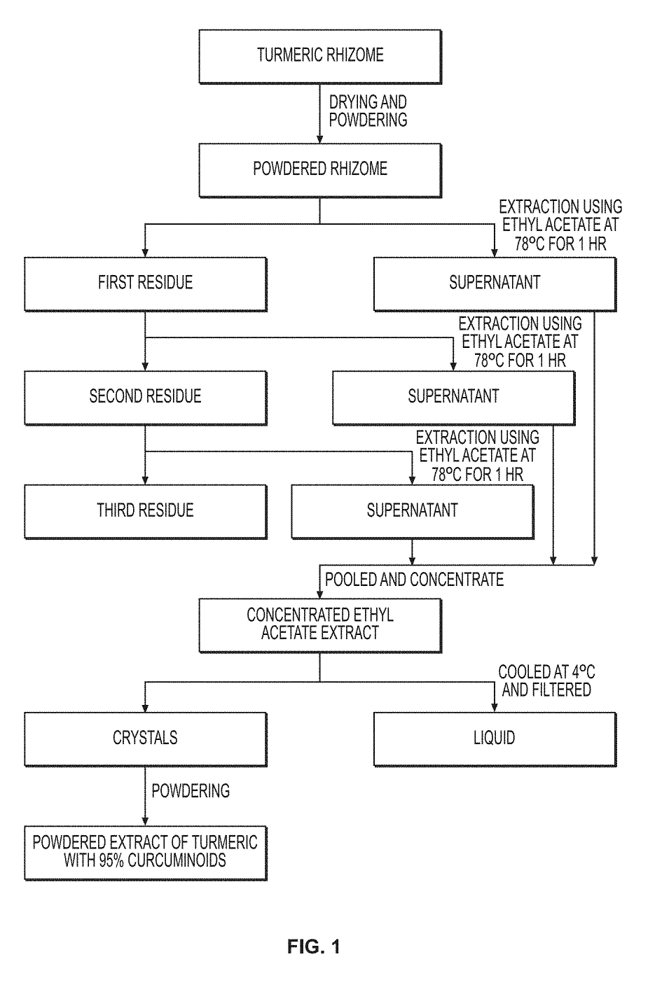

FIG. 1 provides a method for extracting a curcuminoid from turmeric. Rhizomes of turmeric are dried to form dried turmeric rhizomes. The dried turmeric rhizomes are powdered to form powdered turmeric. The powdered turmeric is treated with ethyl acetate at 78° C. for 1 hour to form ethyl acetate extract solution. Ethyl acetate is stripped from the solution to form concentrated ethyl acetate extract. The extract is cooled to about 4° C. to form crystals and a liquid. The liquid includes the essential oil of turmeric and a resin. The crystals are of curcuminoids. The crystals are separated from the liquid to obtain the curcuminoid crystals. The crystals are powdered to form powdered curcuminoid mixture. The powdered curcuminoid mixture is also referred to as regular turmeric extract.

In some embodiments, 95% of the curcuminoid crystals are curcuminoid mixture. The curcuminoid mixture includes curcumin, demethoxycurcumin and bisdemethoxycurcumin.

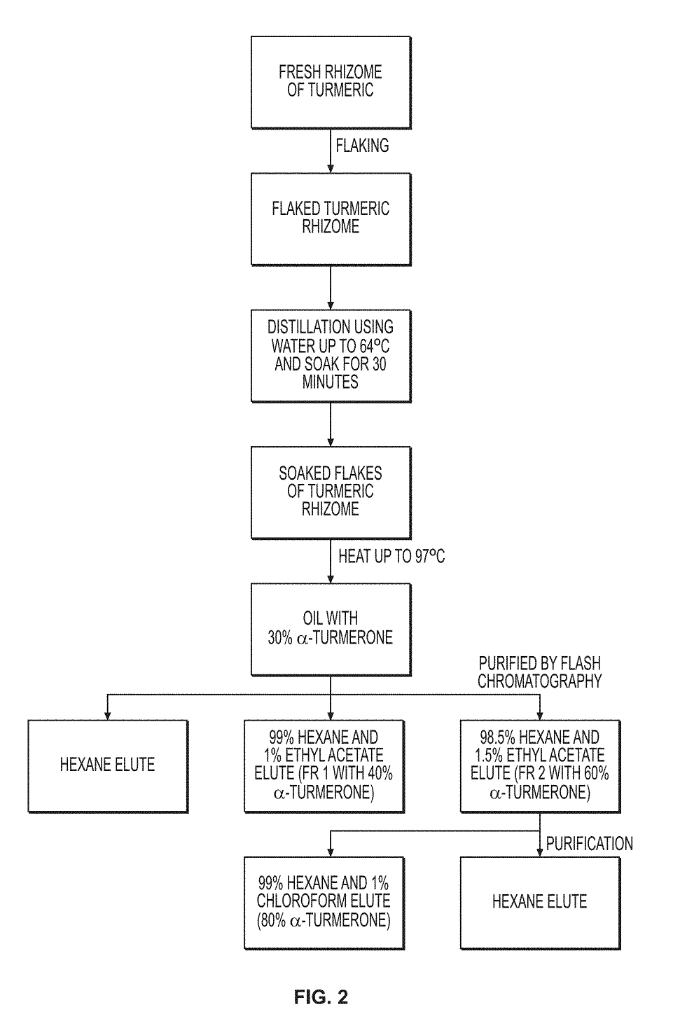

FIG. 2 provides a method for extracting essential oil of turmeric enriched with α-turmerone. Volatile oil of turmeric is isolated by water distillation of fresh turmeric. Flaked turmeric rhizome is distilled using water boiled up to 64° C. and stirred for 15 minutes. Allow to soak for 30 minutes. After soaking, the mixture is heated up to 97° C. and oil formed contains essential oil with 30% α-turmerone. Essential oil with 30% α-turmerone is purified by passing through a column in a flash chromatograph, which was already packed with silica. After loading column is initially eluted with hexane, then eluted with 99% hexane followed by 1% ethyl acetate. 1% ethyl acetate fraction and 99% hexane fractions are collected. 1% ethyl acetate fraction contain 40% α-turmerone, 7% Ar-turmerone and 14% β-turmerone. Then elute with 98.5% hexane followed by 1.5% ethyl acetate. 1.5% ethyl acetate fraction contains 60% α-turmerone, 4% Ar-turmerone and 9% β-turmerone. 1.5° % ethyl acetate fraction is again passed through flash chromatograph then eluted with 99% hexane followed by 1% chloroform. 1% chloroform fraction contains 80% α-turmerone, 1% Ar-turmerone and 4% β-turmerone.

Curcuminoid and the purified essential oil enriched with α-turmerone is blended in a suitable proportion by a process including, suspending the curcuminoid in about 3 to 5 times its quantity of water, mixing in the essential oil, pulverizing in a colloidal mill into slurry, and stripping the slurry off water under heat and vacuum to obtain a powder blend. Five hundred milligram capsules are made from this blend for human consumption.

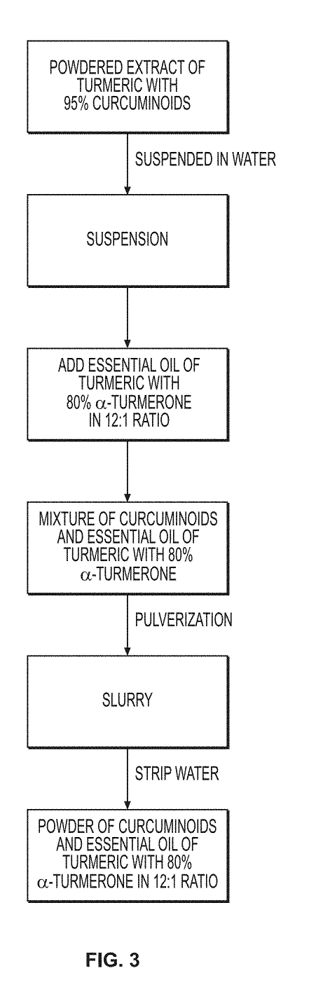

FIG. 3 provides a method of blending curcuminoids with essential oil of turmeric enriched with 80% α-turmerone in 12:1 ratio. Curcuminoid powder is suspended in water to form a suspension. Fraction of essential oil containing 80% α-turmerone is added to the suspension in 12:1 ratio. The mixture is pulverized in a colloidal mill to form slurry. Water is stripped from the slurry under heat and vacuum to form a blend powder of curcuminoids and essential oil of turmeric with 80% α-turmerone.

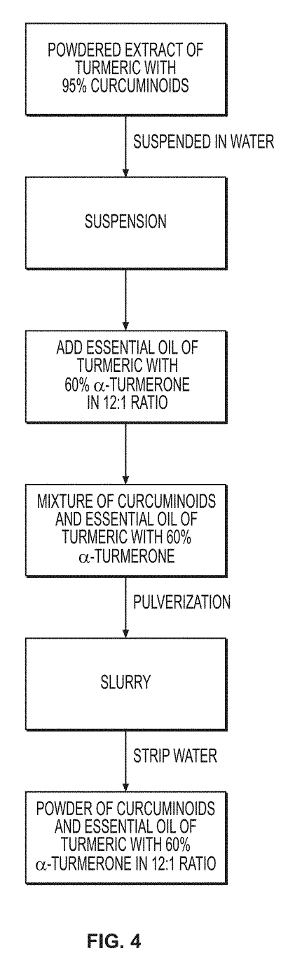

FIG. 4 provides a method of blending curcuminoids with essential oil of turmeric enriched with 60% α-turmerone in 12:1 ratio. Curcuminoid powder is suspended in water to form a suspension. Fraction of essential oil containing 60% α-turmerone is added to the suspension in 12:1 ratio. The mixture is pulverized in a colloidal mill to form slurry. Water is stripped from the slurry under heat and vacuum to form a blend powder of curcuminoids and essential oil of turmeric with 60% α-turmerone.

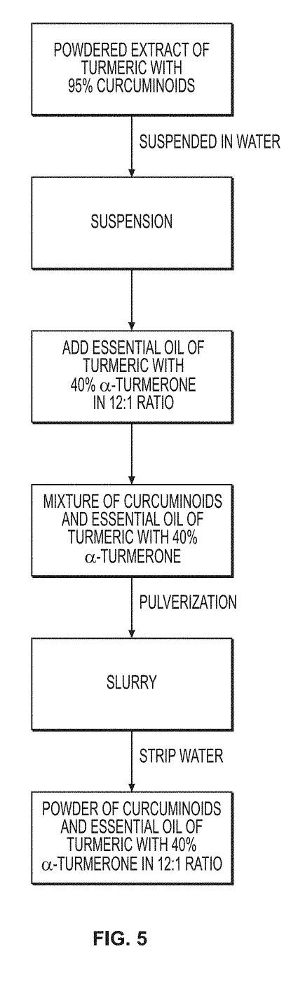

FIG. 5 provides a method of blending curcuminoids with essential oil of turmeric enriched with 40% α-turmerone in 12:1 ratio. Curcuminoid powder is suspended in water to form a suspension. Fraction of essential oil containing 40% α-turmerone is added to the suspension in 12:1 ratio. The mixture is pulverized in a colloidal mill to form slurry. Water is stripped from the slurry under heat and vacuum to form a blend powder of curcuminoids and essential oil of turmeric with 40% α-turmerone.

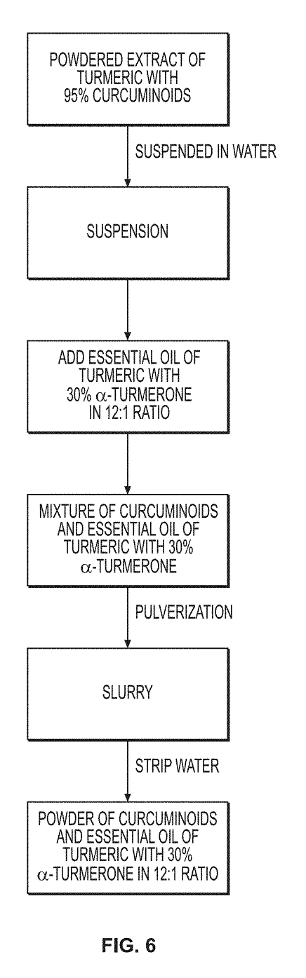

FIG. 6 provides a method of blending curcuminoids with essential oil of turmeric enriched with 30% α-turmerone in 12:1 ratio. Curcuminoid powder is suspended in water to form a suspension. Fraction of essential oil containing 30% α-turmerone is added to the suspension in 12:1 ratio. The mixture is pulverized in a colloidal mill to form slurry. Water is stripped from the slurry under heat and vacuum to form a blend powder of curcuminoids and essential oil of turmeric with 30% α-turmerone.

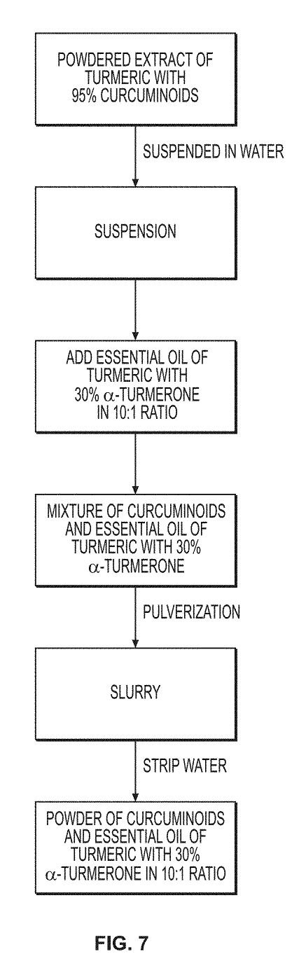

FIG. 7 provides a method of blending curcuminoids with essential oil of turmeric enriched with 30% α-turmerone in 10:1 ratio. Curcuminoid powder is suspended in water to form a suspension. Fraction of essential oil containing 30% α-turmerone is added to the suspension in 10:1 ratio. The mixture is pulverized in a colloidal mill to form slurry. Water is stripped from the slurry under heat and vacuum to form a blend powder of curcuminoids and essential oil of turmeric with 30% α-turmerone.

The curcuminoid mixture is suspended in water to form a suspension. The essential oil of turmeric with alpha turmerone is added to the suspension to form a mixture. The resulting mixture of curcuminoids and essential oil of turmeric is homogenized to obtain slurry. The fine slurry is dried under heat and vacuum to form a powder blend of a composition including the curcuminoid and the essential oil of turmeric enriched with α-turmerone. Drying of the slurry under heat and vacuum can be performed using a vaccumized desolventiser with a stirrer.

In some embodiments the blend obtained after blending curcuminoids and essential oil of turmeric with alpha turmerone in 2:1 to 99:1 is in a powder form. In some embodiment blend obtained after blending curcuminoids and essential oil of turmeric with alpha turmerone in 1:50 to 1:1 is in a liquid form.

Some embodiments provide a method of preparing a composition including a curcuminoid and an essential oil of turmeric.

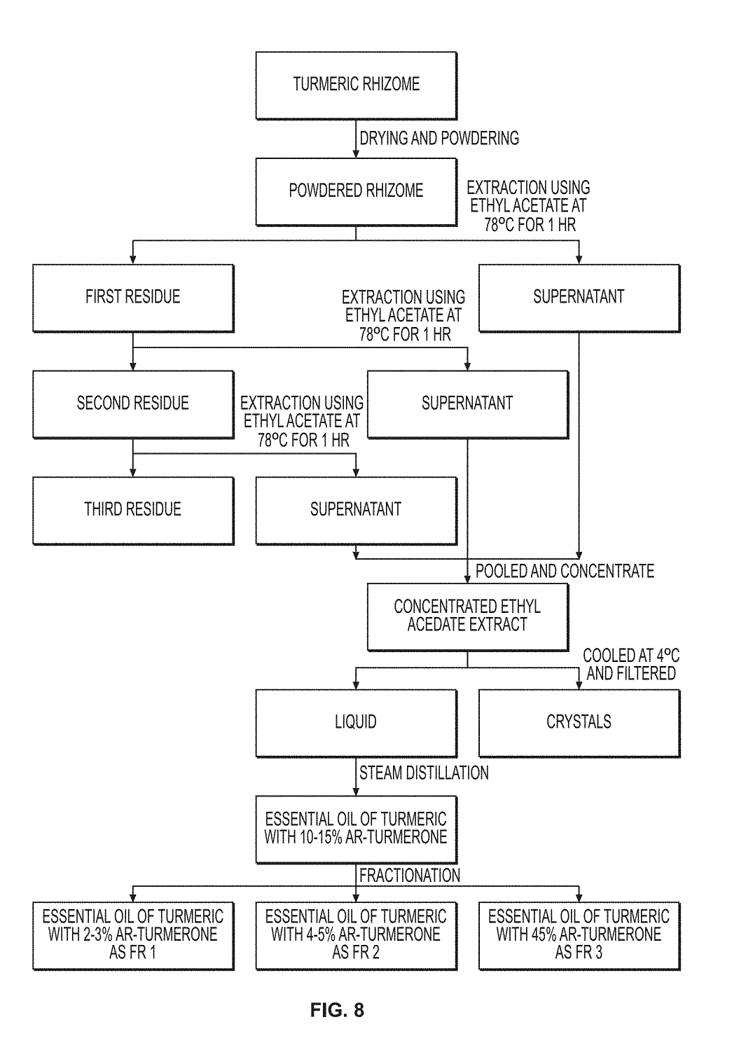

FIG. 8 provides a method of extracting essential oil of turmeric with different percentage of Ar-turmerone. Rhizomes of turmeric are dried to form dried turmeric rhizomes. The dried turmeric rhizomes are powdered to form powdered turmeric. The powdered turmeric is treated with ethyl acetate at 78° C. for 1 hour to form ethyl acetate extract. Ethyl acetate is stripped from the solution to form concentrated ethyl acetate extract. The extract is cooled to about 4° C. to form crystals and a liquid. The liquid includes the essential oil of turmeric and a resin.

The liquid is then steam distilled to isolate essential oil of turmeric with 10-15% Ar-turmerone. After fractionating this oil, essential oil with 45% Ar-turmerone is obtained as fraction 3, essential oil of turmeric with 4-5% Ar-turmerone is obtained as fraction 2 and essential oil of turmeric with 2-3% Ar-turmerone is obtained as fraction 1.

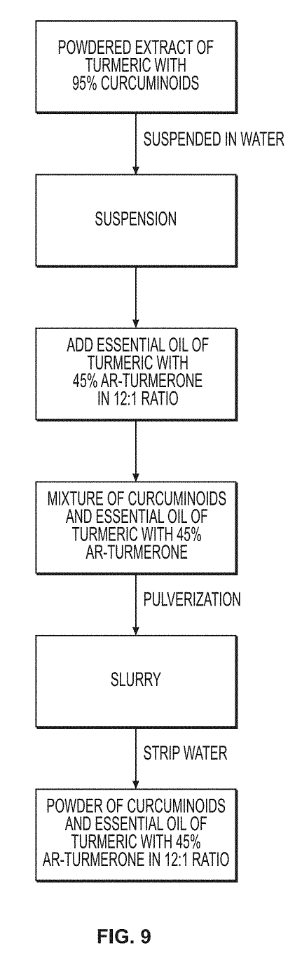

FIG. 9 provides a method of blending curcuminoids with essential oil of turmeric enriched with 45% Ar-turmerone in 12:1 ratio. Curcuminoid powder is suspended in water to form a suspension. Fraction of essential oil containing 45% Ar-turmerone is added to the suspension in 12:1 ratio. The mixture is pulverized in a colloidal mill to form slurry. Water is stripped from the slurry under heat and vacuum to form a blend powder of curcuminoids and essential oil of turmeric with 45% Ar-turmerone.

Some embodiments provide a method of extraction of sesamolin and sesamin from sesame seed.

Sesame (Sesamum indicum) is pulverized and soaked in hexane for 1 hour at 40° C. The suspension is filtered and the residue is again soaked in hexane for 30 min at 40° C. The suspension is filtered and the filtrates are combined and evaporated under reduced pressure to obtain the hexane extract.

The residue of hexane extract is dried at room temperature and extracted with ethyl acetate for 1 hour at 40° C. The suspension is filtered and the filtrates are combined and evaporated under reduced pressure to obtain the ethyl acetate extract.

The residue of ethyl acetate extract is dried at room temperature and extracted with methanol for 1 hour at 40° C. The suspension is filtered and the filtrates are combined and evaporated under reduced pressure to obtain the methanol extract.

Ethyl acetate extract is subjected to silica gel column chromatography using hexane/Ethyl acetate (9:1, v/v), hexane/ethyl acetate (8:1, v/v), hexane/ethyl acetate (4:1, v/v), hexane/ethyl acetate (2:1, v/v), hexane/ethyl acetate (1:1, v/v), ethyl acetate (200 ml each). The active fractions, hexane/ethyl acetate (4:1, 0.22 g), hexane/ethyl acetate (2:1, 0.08 g) and hexane/ethyl acetate (1:1, 0.03 g) fractions were combined for further purification.

The combined fraction is subjected to preparative HPLC. Sesamin and sesamolin are observed at retention times of 7.5 and 8.5 min, respectively. The compounds are obtained as white amorphous powders.

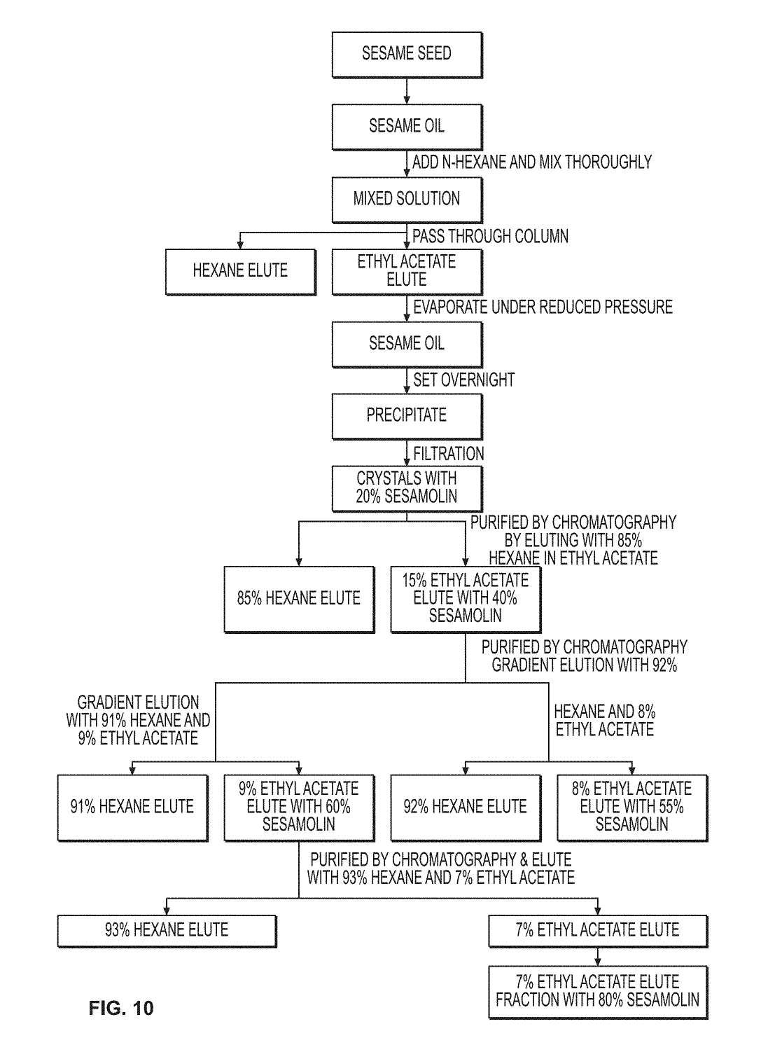

FIG. 10 provides a method of extracting sesame seed with different ratios of sesamin and sesamolin. Sesame seed is expelled to form sesame oil. Sesame oil is passed through adsorption resin column and eluted with hexane and ethyl acetate. Ethyl acetate is removed from the ethyl acetate fraction to form oil. Oil is set overnight to precipitate the formed crystals. The precipitant is collected by filtration contain 20% sesamolin.

Sesame seed extract with 20% sesamolin is further purified by flash chromatography. Impregnated sesame seed extract is transferred in to the top of the column in a flash chromatograph. After loading the sample, column is initially eluted with 85% hexane in Ethyl acetate. 15% ethyl acetate fraction contains 40% sesamolin. Sesame seed extract with 40% sesamolin is impregnated with silica gel in 2:1 ratio. The impregnated sample is transferred in to the top of the column in a flash chromatograph. After loading the column is initially eluted by gradient elution with 92% hexane and 8% ethyl acetate (Fm1) & followed by 91% hexane & 9% ethyl acetate (Fm2). Fm1& Fm2 are collected. 8% ethyl acetate fraction contains 55% sesamolin and 9% ethyl acetate fraction contains 60% sesamolin. 9% ethyl acetate fraction is impregnated and transferred in to the top of the column in a flash chromatograph. After loading the sample, column is initially eluted with 93% hexane and 7% ethyl acetate. 7% ethyl acetate fraction contains 80% sesamolin.

Some embodiments provide method of preparation of essential oil of turmeric enriched with α-turmerone and sesamolin enriched extract in different ratios.

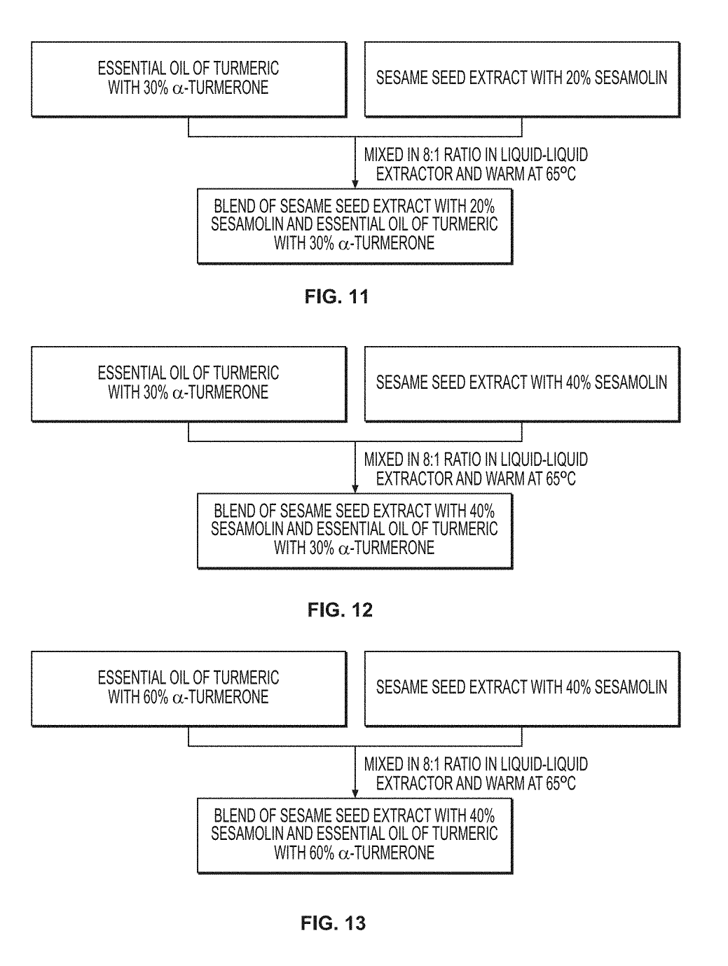

FIG. 11 provides a method of blending essential oil of turmeric enriched with 30% α-turmerone and 20% sesamolin in 8:1 ratio. Essential oil of turmeric enriched with 30% α-turmerone and 20% sesamolin is mixed in 8:1 ratio in a liquid-liquid extractor. Warmed at 65° C. to form a blend of sesame seed extract with 20% sesamolin and essential oil of turmeric with 30% α-turmerone.

FIG. 12 provides a method of blending essential oil of turmeric enriched with 30% α-turmerone and 40% sesamolin in 8:1 ratio. Essential oil of turmeric enriched with 30% α-turmerone and 40% sesamolin is mixed in 8:1 ratio in a liquid-liquid extractor. Warmed at 65° C. to form a blend of sesame seed extract with 40% sesamolin and essential oil of turmeric with 30% α-turmerone.

FIG. 13 provides a method of blending essential oil of turmeric enriched with 60% α-turmerone and 40% sesamolin in 8:1 ratio. Essential oil of turmeric enriched with 60% α-turmerone and 40% sesamolin is mixed in 8:1 ratio in a liquid-liquid extractor. Warmed at 65° C. to form a blend of sesame seed extract with 40% sesamolin and essential oil of turmeric with 60% α-turmerone.

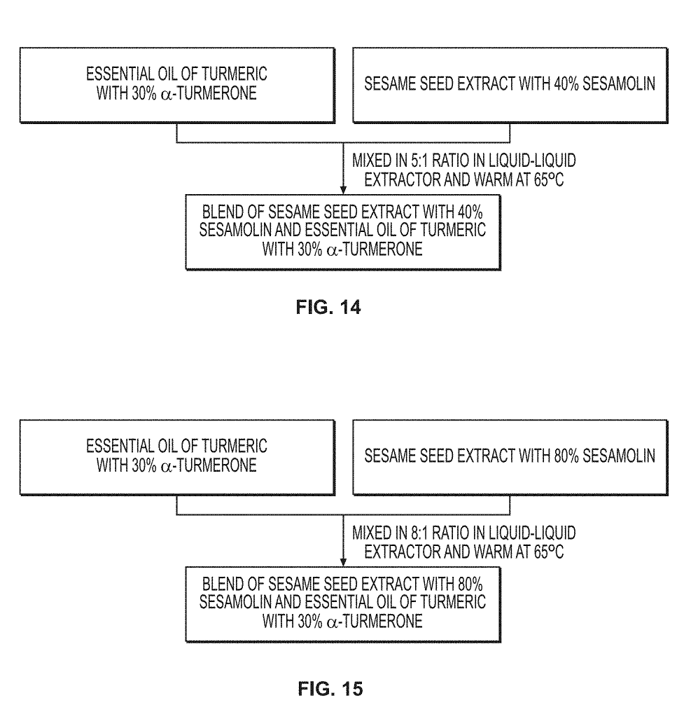

FIG. 14 provides a method of blending essential oil of turmeric enriched with 30% α-turmerone and 40% sesamolin in 5:1 ratio. Essential oil of turmeric enriched with 30% α-turmerone and 40% sesamolin is mixed in 5:1 ratio in a liquid-liquid extractor. Warmed at 65° C. to form a blend of sesame seed extract with 40% sesamolin and essential oil of turmeric with 30% α-turmerone.

FIG. 15 provides a method of blending essential oil of turmeric enriched with 30% α-turmerone and 80% sesamolin in 8:1 ratio. Essential oil of turmeric enriched with 30% α-turmerone and 80% sesamolin is mixed in 8:1 ratio in a liquid-liquid extractor. Warmed at 65° C. to form a blend of sesame seed extract with 80% sesamolin and essential oil of turmeric with 30% α-turmerone.

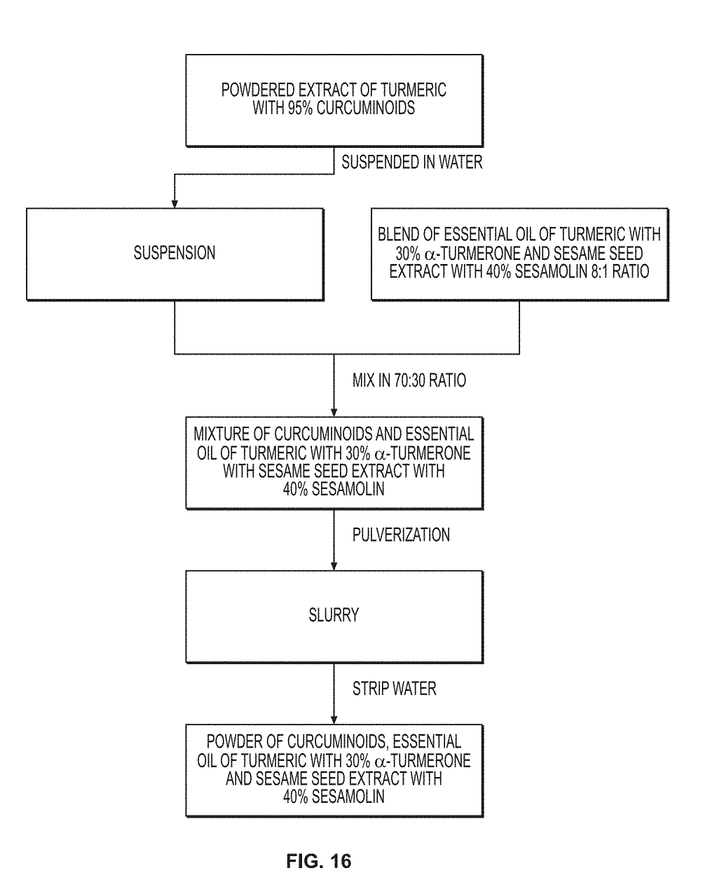

FIG. 16 provides a method of preparation of curcuminoid and combination of essential oil of turmeric enriched with α-turmerone and sesamolin in 70:30 ratio. Curcuminoid and combination of purified essential oil enriched with α-turmerone and sesamolin enriched extract is blended in 70:30 proportion by a process including, suspending the curcuminoid in about 3 to 5 times its quantity of water, mixing 8:1 ratio of essential oil and sesamolin enriched extract, pulverizing in a colloidal mill into slurry, and stripping the slurry off water under heat and vacuum to obtain a powder blend.

The disclosure provides a method of treatment of brain related disorders by orally administering a blend of curcuminoids, purified essential oil of turmeric with α-turmerone and sesame seed extract with sesamolin and sesamin.

Some embodiments provide a dosage form or delivery system including capsule, tablet, granule, sachet, powder, paste, infusion, ampoule, solution, suspension, emulsion, pills or cream.

These delivery systems may require excipients selected from the group consisting of a disintegrant, diluents, binders, fillers, a carrier, adsorbents, emulsifiers, lubricants, stabilizing agents, antiadherents, galidants, antioxidants and mixtures thereof.

The disclosure provides a method of treatment of brain related disorders by orally administering a blend of curcuminoids, purified essential oil of turmeric with α-turmerone and sesame seed extract with sesamolin and sesamin.

In some embodiments provide a composition containing sesamolin in different ratios and its β-secretase inhibitory activity. From example 17, beta-secretase activity is increasing as the concentration of sesamolin is increased. Sesame seed extract with 80% sesamolin showed the most potent inhibitory activity (42.86%). Sesame seed extract with 40%, 55% and 60% sesamolin shows the β-secretase inhibitory activity 24.14%, 26.86% and 28.93% respectively. Sesame seed extract with 20% sesamolin shows least inhibitory activity (8.3%). In another embodiment β-Secretase inhibitory activities of essential oil of turmeric (EOT) with different percentage of α-turmerone is provided (example 18). From the result it is clear that essential oil of turmeric enriched with α-turmerone shows inhibitory assay against beta-secretase. Essential oil of turmeric enriched with 80% α-turmerone shows 44.6% inhibition. Essential oil of turmeric enriched with 60% α-turmerone shows 38.4% inhibition compared to essential oil of turmeric with 30% α-turmerone (7.2% inhibition).

β-Secretase inhibitory activities of combination of essential oil of turmeric (EOT) enriched with α-turmerone and sesamin-sesamolin in different ratios shows different inhibitory activity (example 19). Combination of essential oil of turmeric enriched with 30% α-turmerone and sesame seed extract with 80% sesamolin in 8:1 ratio significantly higher beta-secretase inhibitory activity (53.4%) compared with combination of essential oil of turmeric enriched with 30% α-turmerone and 20% sesamolin in 8:1 ratio (24.2% Inhibition). Essential oil of turmeric enriched with 60% α-turmerone and 40% sesamolin blended in 8:1 ratio shows 35.6% inhibition and essential oil of turmeric enriched with 30% α-turmerone and 40% sesamolin blended in 8:1 ratio shows 26.1% inhibition.

Some embodiments include a composition having a curcuminoid and an added amount of essential oil of turmeric enriched with α-turmerone, wherein the essential oil of turmeric enriched with α-turmerone has greater β-secretase inhibitory activity as compared to the β-secretase inhibitory activity of curcumin alone. From the study results (example 20) combination of curcuminoids and essential oil of turmeric enriched with 80% α-turmerone in 12:1 ratio shows higher beta-secretase inhibitory activity (45.9%) compared to curcuminoid alone (8.9% inhibition). Combination of curcuminoids and essential oil of turmeric enriched with 60% α-turmerone in 12:1 ratio shows 38.4% inhibition and curcuminoids and essential oil of turmeric enriched with 40% α-turmerone in 12:1 ratio shows 29.2% inhibition. Combination of curcuminoids and essential oil of turmeric enriched with 30% α-turmerone in 10:1 ratio shows 17.4% inhibition.

In one embodiment β-Secretase inhibitory activities of combination of curcuminoids, essential oil of turmeric (EOT) enriched with α-turmerone and sesame seed extract is provided. From example 22 it is clear that curcuminoids blended with 8:1 ratio of EOT with 30% α-turmerone and 40% sesamolin in 70:30 ratio shows higher beta-secretase inhibitory activity (48.6%) compared to curcuminoid alone (9.5%). EOT with 30% α-turmerone and 40% sesamolin in 8:1 ratio shows 27.4% inhibition.

In one embodiment the beta secretase inhibitory activity of curcuminoids, essential oil of turmeric enriched with alpha turmerone and sesamolin in different dosages are studied. In one embodiment curcuminoids at different dosages (50 μg/ml, 46 μg/ml and 45 μg/ml) is found to have beta secretase inhibitory activity. In another embodiment essential oil of turmeric enriched with alpha turmerone at different dosages (50 μg/ml, 44 μg/ml, 41.6 μg/ml, 3.8 μg/ml and 4.5 μg/ml) is found to have beta secretase inhibitory activity. In another embodiment sesamolin at different dosages (50 μg/ml, 5.5 μg/ml, and 8.3 μg/ml) is found to have beta secretase inhibitory activity.

Another embodiment provides a composition to enhance the availability of α-turmerone, β-turmerone. Ar-turmerone, sesamin and sesamolin in brain and serum. From example 23 after administering essential oil of turmeric with 40% α-turmerone:sesame seed extract with 25% sesamolin in 5:1 ratio in mice shows the availability of sesamin, sesamolin, α-Turmerone, (3-turmerone and α-turmerone in the brain and serum. At 25 mg/Kg dose, sesamin and sesamolin are detected at 15.9 and 38.9 ng/ml in the serum (Total lignans 54.8 ng/ml). 25.62 and 50.19 ng/mg (Total lignans 75.81) in the brain. At 25 mg/Kg dose, α-turmerone, f-turmerone and Ar-turmerone are detected at 26.2, 10.77 and 6.89 ng/ml in the serum, 19.8, 8.6 and 4.8 ng/mg in the brain. After administering sesamolin 25% at 25 mg/Kg dose, sesamin and sesamolin is detected at 5.2 and 9.8 ng/ml in the serum and 8.5 and 12.5 ng/mg in the brain. The availability of sesamolin in brain is 4 times higher in groups fed with essential oil of turmeric with 40% α-turmerone:sesame seed extract with 25% sesamolin in 5:1 ratio compared to groups fed with sesamolin 25% alone. α-turmerone availability in brain is 2 times higher in groups fed with essential oil of turmeric with 40% α-turmerone:sesame seed extract with 25% sesamolin in 5:1 ratio compared to groups fed with essential oil of turmeric with 40% α-turmerone alone.

Another embodiment provides a composition to enhance the availability of curcumin, demethoxycurcumin and bisdemethoxycurcumin in plasma by administering a combination of curcuminoid and essential oil of turmeric enriched with α-turmerone in 12:1 ratio. The results from example 24 indicate that curcumin, demethoxycurcumin and bisdemethoxy curcumin is detected in animals fed with curcuminoids and essential oil of turmeric enriched with 60% α-turmerone in 12:1 ratio. Animals fed with curcuminoids and essential oil of turmeric enriched with 45% Ar-tumerone in 12:1 ratio (Group 3) shows detection of curcumin only in plasma. Demethoxycurcumin and bisdemethoxy curcumin is not detected in animals fed with curcuminoids and essential oil of turmeric enriched with 45% Ar-tumerone in 12:1 ratio. Animals fed with curcuminoid mixture alone shows low detection of curcumin in plasma.

Another embodiment provides a composition to enhance the availability of curcumin, demethoxycurcumin and bisdemethoxycurcumin in brain by administering a combination of curcuminoid and essential oil of turmeric enriched with α-turmerone in 12:1 ratio. The results from example 25 indicate that curcumin, demethoxycurcumin and bisdemethoxy curcumin is detected in brain of animals fed with curcuminoids and essential oil of turmeric enriched with 60% α-turmerone in 12:1 ratio. α-turmerone, β-turmerone and Ar-turmerone is also detected in brain of group 2 animals fed with curcuminoids and essential oil of turmeric enriched with 60% α-turmerone in 12:1 ratio. Animals fed with curcuminoids and essential oil of turmeric enriched with 45% Ar-tumerone in 12:1 ratio (Group 3) shows small detection of curcumin, demethoxycurcumin, α-turmerone and Ar-turmerone in brain. Demethoxycurcumin and bisdemethoxycurcumin is not detected in brain of rats fed with curcuminoids alone but a slight detection of curcumin is only found.

### EXAMPLES

Following examples 1 to 16 illustrate the method of preparation of the natural composition comprising of turmeric and sesame seed extract according to different embodiment under the invention. Example 17 to 25 illustrate the method of treatment of brain related disorders by orally administering a blend of curcuminoids, purified essential oil of turmeric with α-turmerone and sesame seed extract with sesamolin and sesamin disclosed under different embodiment under the invention.

### Example 1

Method of Preparation of Regular Turmeric Extract (95% Curcuminoid Mixture)

The rhizomes of turmeric (300 Kg) were dried. The dried turmeric rhizomes were powdered to form powdered turmeric. The powdered turmeric was treated with ethyl acetate (900 L) to form a solution. The extraction was carried out at 78° C. temperature for 1 hr. After initial extraction, the extraction process was repeated 4 more times and the resultant solution was filtered and the solvent was stripped from the filtered solution to form an extract. This extract was cooled to about 4° C. to obtain crystals of curcuminoid (12 Kg) and a liquid. The crystals of curcuminoid were isolated from the liquid by filtration. The crystals included a mixture of curcumin, demethoxycurcumin and bisdemethoxycurcumin. 95% of the crystals were composed of the mixture of curcumin, demethoxycurcumin and bisdemethoxycurcumin. The crystals were powdered to form powdered curcuminoid mixture. The powdered curcuminoid mixture was also referred to as regular turmeric extract contain Curcumin 73%, Demethoxycurcumin 16.2% and Bisdemethoxycurcumin 5.8%.

### Example 2

Method of Preparation of Essential Oil of Turmeric Enriched with α-Turmerone

Fresh rhizomes of turmeric (500 Kg) were cleaned. The cleaned turmeric rhizomes were flaked. The flaked turmeric was distilled using water (2500 L) in a vessel. Water was boiled up to 64° C. under stirring condition. Flaked turmeric was added in to the boiled water and stirred for 15 minutes. Then allowed for soaking for 30 minutes. After soaking, the mixture was heated up to 97° C. Distillation started and oil along with water was collected in a separator. Floating oils were collected and contains 30% α-turmerone, 17% Ar-turmerone and 16% β-turmerone. [Sample 1]

The essential oil of turmeric was further purified by flash chromatography. The silica gel particles of size about 40-63 μm are loaded in to a KP-SIL50 g SNAP cartridge & the column was primed (wet) with hexane. Before loading to the column, essential oil of turmeric was impregnated with silica gel in 1:1 ratio. The impregnated oil was transferred in to the samplet of the cartridge in a flash chromatograph. After sample loading, column was initially eluted with 2 column volume of hexane followed by a gradient elution with 6 column volume of 99% hexane and 1% ethyl acetate (Fr1). After that column eluted with 8 column volume of 98.5% hexane and 1.5% ethyl acetate (Fr 2). Fraction1 contains 40% α-turmerone, 7% Ar-turmerone and 14% β-turmerone [Sample 2] and fr 2 contains 60% α-turmerone, 4% Ar-turmerone and 9% β-turmerone [Sample 3].

For further purification, sample 3 was impregnated with silica gel in 1:1 ratio. The impregnated oil was transferred in to the samplet of the column in a flash chromatograph, which was already packed with silica. Before loading the column, the column was wetted with hexane. After sample loading the column was initially eluted with 2 column volume of hexane followed by a gradient elution with 4 column volume of 99% hexane and 1% chloroform. 1% chloroform and 99% hexane fractions were collected. 1% chloroform fraction contains 80% α-turmerone, 1% Ar-turmerone and 4% β-turmerone [Sample 4].

### Example 3

Method of Preparation of Combination of Curcuminoids and Essential Oil of Turmeric with 80% α-Turmerone in 12:1 Ratio.

The curcuminoid powder prepared as per Example 1 (3.5 Kg) was suspended in water (15 L) to form a suspension. Fraction of essential oil containing 80% α-turmerone (sample 4 prepared as per Example 2 (0.29 Kg) was added to the suspension in 12:1 ratio. The mixture was pulverized in a colloidal mill to form slurry. Water was stripped from the slurry under heat and vacuum to form a blend (3.8 Kg). Blend was a powder containing Curcumin 67.38%, Demethoxycurcumin 14.95% and Bisdemethoxycurcumin 5.35%.

### Example 4

Method of Preparation of Combination of Curcuminoids and Essential Oil of Turmeric with 60% α-Turmerone in 12:1 Ratio.

The curcuminoid powder prepared as per Example 1 (3.5 Kg) was suspended in water (15 L) to form a suspension. Fraction of essential oil containing 60% α-turmerone sample 3 prepared as per Example 2 (0.29 Kg) was added to the suspension in 12:1 ratio. The mixture was pulverized in a colloidal mill to form slurry. Water was stripped from the slurry under heat and vacuum to form a blend (3.8 Kg). Blend was a powder containing Curcumin 67.38%, Demethoxycurcumin 14.95% and Bisdemethoxycurcumin 5.35%.

### Example 5

Method of Preparation of Combination of Curcuminoids and Essential Oil of Turmeric with 40% α-Turmerone in 12:1 Ratio.

The curcuminoid powder prepared as per Example 1 (3.5 Kg) was suspended in water (15 L) to form a suspension. Fraction of essential oil containing 40% α-turmerone sample 2 prepared as per Example 2 (0.29 Kg) was added to the suspension in 12:1 ratio. The mixture was pulverized in a colloidal mill to form slurry. Water was stripped from the slurry under heat and vacuum to form a blend (3.8 Kg). Blend was a powder containing Curcumin 67.38%, Demethoxycurcumin 14.95% and Bisdemethoxycurcumin 5.35%.

### Example 6

Method of Preparation of Combination of Curcuminoids and Essential Oil of Turmeric with 30% α-Turmerone in 12:1 Ratio.

The curcuminoid powder prepared as per Example 1 (3.5 Kg) was suspended in water (15 L) to form a suspension. Fraction of essential oil containing 30% α-turmerone sample 1 prepared as per Example 2 (0.29 Kg) was added to the suspension in 12:1 ratio. The mixture was pulverized in a colloidal mill to form slurry. Water was stripped from the slurry under heat and vacuum to form a blend (3.8 Kg). Blend was a powder Curcumin 67.38%.

Demethoxycurcumin 14.95% and Bisdemethoxycurcumin 5.35%.

### Example 7

Method of Preparation of Combination of Curcuminoids and Essential Oil of Turmeric with 30% α-Turmerone in 10:1 Ratio.

The powdered curcuminoid mixture prepared as per Example 1 (2.7 Kg) was suspended in water (12 L) to form a suspension. Fraction of essential oil of turmeric containing 30% α-turmerone sample 1 prepared as per Example 2 (0.27 Kg) was added to the suspension in 10:1 ratio. The mixture was pulverized in a colloidal mill to form slurry. Water was stripped from the slurry under heat and vacuum to form a blend (3 Kg). Blend was a powder Curcumin 66.36%, Demethoxycurcumin 14.72% and Bisdemethoxycurcumin 5.27%.

### Example 8

Method of Preparation of Essential Oil of Turmeric with 45% Ar-Turmerone

The rhizomes of turmeric (500 Kg) were dried. The dried turmeric rhizomes were powdered to form powdered turmeric. The powdered turmeric was treated with ethyl acetate (1500 L) to form a solution. The extraction was carried out at 78° C. temperature for 1 hr. After initial extraction, the extraction process was repeated 4 more times and the resultant solution was filtered and the solvent was stripped from the filtered solution to form an extract. This extract was cooled to about 4° C. to obtain crystals of curcuminoid mixture (20 Kg) and a liquid. The crystals of curcuminoid were isolated from the liquid by filtration.

The remaining liquid included the essential oil of turmeric and a resin. The liquid was then steam distilled to isolate essential oil of turmeric with 10-15% Ar-turmerone (25 Kg). After fractionating this oil, essential oil with 45% Ar-turmerone (7.5 Kg) was obtained as fraction 3, essential oil of turmeric with 4-5% Ar-turmerone (8.3) was obtained as fraction 2 and essential oil of turmeric with 2-3% Ar-turmerone (9.3 Kg) was obtained as fraction 1.

### Example 9

Method of Preparation of Combination of Curcuminoids and Essential Oil of Turmeric with 45% Ar-Turmerone in 12:1 Ratio.

The curcuminoid powder prepared as per Example 1 (3.5 Kg) was suspended in water (15 L) to form a suspension. Fraction of essential oil containing 45% Ar-turmerone prepared as per Example 8 (0.29 Kg) was added to the suspension in 12:1 ratio. The mixture was pulverized in a colloidal mill to form slurry. Water was stripped from the slurry under heat and vacuum to form a blend (3.8 Kg). Blend was a powder Curcumin 67.38%, Demethoxycurcumin 14.95% and Bisdemethoxycurcumin 5.35%.

### Example 10

Method of Preparation of Extract of Sesame Seed with Different Ratios of Sesamin and Sesamolin.

2000 Kg of sesame seed was expelled to form sesame oil (1000 Kg). 1000 L of n-hexane, 1000 Kg of sesame oil was added and thoroughly mixed to obtain a mixed solution. The mixture was passed through the amber 200 Column. Before passing through the column, the column was washed with hexane. After loading, column was initially eluted with hexane followed by ethyl acetate. Hexane fraction and ethyl acetate fractions were collected. The solvent was removed from the ethyl acetate fraction by evaporation under a reduced pressure to obtain oil. Then, the oil was set overnight to precipitate the formed crystals. The precipitant was collected by filtration. Crystals contain sesamin content 60% and the sesamolin content was 20%. [Sample 1, Yield 9 Kg].

Sample 1 was further purified by flash chromatography. The silica gel particles size about 40-63 μm are loaded in to a KP-SIL50 g SNAP cartridge & the column was prime (wet) with 85% hexane in ethyl acetate. Before loading to the column, sample 1 was solubilised in ethyl acetate and impregnated with silica gel in 2:1 ratio. The impregnated sample 1 was transferred in to the top of the column in a flash chromatograph. After loading the sample, column was initially eluted with 4 column volume of 85% hexane in EA. 15% ethyl acetate fraction and 85% hexane fractions were collected. 15% ethyl acetate fraction contains 40% sesamolin and 40% sesamin [Sample 2, Yield 2.7 Kg].

Sample 2 was impregnated with silica gel in 2:1 ratio. The impregnated sample 2 was transferred in to the top of the column in a flash chromatograph, which was already packed with silica. Before loading the column, the column was washed with 92% hexane in Ethyl acetate. After loading the column was initially 6 column volume of gradient elution with 92% hexane and 8% ethyl acetate (Fm1) & followed by 4 column volume of 91% hexane & 9% ethyl acetate (Fm2). Fm1& Fm2 were collected. 8% ethyl acetate fraction contains 25% sesamin and 55% sesamolin [Sample 3, yield 1.8 Kg]. 9% ethyl acetate fraction contains 20% sesamin and 60% sesamolin [Sample 4, Yield 0.72 Kg]

Sample 4 was impregnated with silica gel in 2:1 ratio. The impregnated sample 4 was transferred in to the top of the column in a flash chromatograph, which was already packed with silica. Before loading the column, the column was wetted with 93% hexane in Ethyl acetate. After loading the sample, column was initially eluted with 6 column volume of 93% hexane and 7% ethyl acetate. 7% ethyl acetate and 93% hexane fractions were collected. 7% ethyl acetate fraction contains 80% sesamolin [Sample 5, Yield 0.4 Kg].

Method of Extraction of Sesame Seed

Sesame (Sesamum indicum) was pulverized and soaked into 5 volumes/weight of hexane for 1 hour at 40° C. The suspension was filtered and the residue was again soaked into the same volume of hexane for 30 min at 40′C. The suspension was filtered and the filtrates were combined and evaporated under reduced pressure to obtain the hexane extract.

The residue of hexane extract was dried at room temperature and extracted with ethyl acetate for 1 hour at 40° C. The suspension was filtered and the residue was again soaked into the same volume of ethyl acetate for 30 min at 40° C. The suspension was filtered and the filtrates were combined and evaporated under reduced pressure to obtain the ethyl acetate extract.

The residue of ethyl acetate extract was dried at room temperature and extracted with methanol for 1 hour at 40° C. The suspension was filtered and the residue was again soaked into the same volume of methanol for 30 min at 40° C. The suspension was filtered and the filtrates were combined and evaporated under reduced pressure to obtain the methanol extract.

Ethyl acetate extract was subjected to silica gel column chromatography using hexane/Ethyl acetate (9:1, v/v) (Yield: 3.5 g), hexane/ethyl acetate (8:1, v/v) (Yield: 0.03 g), hexane/Ethyl acetate (4:1, v/v) (Yield: 0.22 g), hexane/ethyl acetate (2:1, v/v) (Yield: 0.08 g), hexane/ethyl acetate (1:1, v/v) (Yield: 0.03 g), ethyl acetate (200 ml each) (Yield: 0.87 g). The active fractions, hexane/ethyl acetate (4:1, 0.22 g), hexane/ethyl acetate (2:1, 0.08 g) and hexane ethyl acetate (1:1, 0.03 g) fractions were combined for further purification.

The combined fraction was subjected to preparative HPLC under the conditions in the column: L-column ODS (20 i.d.×250 mm), mobile phase: water/acetonitrile (MeCN) (1:9, v/v, isocratic), flow rate: 18.9 ml/min, detection: UV 286 nm. Sesamin and sesamolin were observed at retention times of 7.5 and 8.5 min. respectively. The compounds were obtained as white amorphous powders.

### Example 11

Method of Preparation of Combination of Essential Oil of Turmeric Enriched with α-Turmerone (30%) and Sesamolin (20%) in 8:1 Ratio

The essential oil of turmeric enriched with 30% α-turmerone (0.88 Kg) (sample 1 prepared as per Example 2) and 20% sesamolin (0.11 Kg) sample 1 prepared as per example 10 was mixed in 8:1 ratio in a liquid-liquid extractor. Warmed at 65° C. to form a blend containing Sesamin 6.66% sesamolin 2.22% and 26.66% α-turmerone.

### Example 12

Method of Preparation of Combination of Essential Oil of Turmeric Enriched with α-Turmerone (30%) and Sesamolin (40%) in 8:1 Ratio.

The essential oil of turmeric enriched with 30% α-turmerone (0.88 Kg) (sample 1 prepared as per Example 2) and 40% sesamolin (0.11 Kg) sample 2 prepared as per example 10 was mixed in 8:1 ratio in a liquid-liquid extractor. Warmed at 65° C. to form a blend containing sesamin 4.44% sesamolin 4.44% and 26.66% α-turmerone.

### Example 13

Method of Preparation of Combination of Essential Oil of Turmeric Enriched with α-Turmerone (60%) and Sesamolin (40%) in 8:1 Ratio.

The essential oil of turmeric enriched with 60% α-turmerone (0.88 Kg) (sample 3 prepared as per Example 2) and 40% sesamolin (0.11 Kg) sample 2 prepared as per example 11 was mixed in 8:1 ratio in a liquid-liquid extractor. Warmed at 65° C. to form a blend containing sesamin 4.44% sesamolin 4.44% and 53.3% α-turmerone.

### Example 14

Method of Preparation of Combination of Essential Oil of Turmeric Enriched with α-Turmerone (30%) and Sesamolin (40%) in 5:1 Ratio.

The essential oil of turmeric enriched with 30% α-turmerone (0.83 Kg) (sample 2 prepared as per Example 2) and 40% sesamolin (0.16 Kg) sample 2 prepared as per example 10 was mixed in 5:1 ratio in a liquid-liquid extractor. Warmed at 65° C. to form a blend containing sesamin 6.66%, sesamolin 6.66% and 25% α-turmerone.

### Example 15

Method of Preparation of Combination of Essential Oil of Turmeric Enriched with α-Turmerone (30%) and Sesamolin 80% in 8:1 Ratio.

The essential oil of turmeric enriched with 30% α-turmerone (0.88 Kg) (sample 2 prepared as per Example 2) and 80% sesamolin (0.11 Kg) sample 5 prepared as per example 14 was mixed in 8:1 ratio in a liquid-liquid extractor. Warmed at 65° C. to form a blend (1 Kg) containing sesamolin 8.88% and 26.66% α-turmerone.

### Example 16

Method of Preparation of Combination of Curcuminoid and 8:1 Combination of Essential Oil of Turmeric Enriched with 30% α-Turmerone and 40% Sesamolin in 70:30 Ratio.

The curcuminoid powder prepared as per Example 1 (0.7 Kg) was suspended in water (3 L) to form a suspension. Essential oil of turmeric enriched with 30% α-turmerone and 40% sesamolin blended in 8:1 ratio prepared as per Example 12 (0.3 Kg) was added to the suspension in 70:30 ratio. The mixture was pulverized in a colloidal mill to form fine slurry. Water was stripped from the slurry under heat and vacuum to form a blend (1 Kg). Blend was a powder containing curcumin 51.1%, demethoxycurcumin 11.34% and bisdemethoxycurcumin 4.06%. α-turmerone in the blend was 7.99%, sesamin 1.33% and sesamolin 1.33%.

### Example 17

β-Secretase Inhibitory Activities of Sesamolin in Different Ratios from Sesame Seed Extract.

### Material Used

β-Secretase (human, recombinant), A fluorescent substrate [MOCAc-Ser-Glu-Val-Asn-Leu-Asp-Ala-Glu-Phe-Arg-Lys (Dnp) Arg-Arg-NH2] and a β-secretase inhibitor[Lys-Thr-Glu-Glu-Ile-Ser-Glu-Val-Asn-Sta-Val-Ala-Glu-Phe(Sta:(3S,4S)-4-amino-3-hydroxy-6-methylheptanoic acid)] Other reagents used in this study were analytical grade. Sesamin-sesamolin in different ratios prepared as per examples 10.

Procedure

Samples in a dimethylsulfoxide solution (2 μl), at an appropriate concentration, were diluted with 78 μl of assay buffer (20 mM acetate buffer, pH=4.5 containing 1% triton X-100) in a 96-well microplate. 10 μl of β-secretase solution in assay buffer (17.4 μg protein/ml) was added to the diluted solution, and incubated at 37° C. for 20 min. After incubation, 10 μl of substrate solution with assay buffer (0.1 mM) was added and incubated at 37° C. for 2 h. Fifty μl of reaction solution was then added to 450 μl of 2.5 M sodium acetate solution to terminate the reaction. The diluted solution was analyzed by HPLC applying the conditions listed below. Column: L-column ODS (Chemicals Evaluation and Research Institute, Tokyo, Japan, 4.6 i.d.×250 mm), mobile phase; 0.1% (v/v) formic acid/acetonitrile (0.1% (v/v) formic acid); 0 min (9:1), 17.5 min (5.5:4.5), 17.6 min (0.5:9.5), 22.5 min (0.5:9.5), column temperature: 40° C., flow rate; 1 ml/min, detection; fluorescent of excitation at 325 nm and emission at 395 nm, injection volume; 20 μl. The peak area of the peak derived from the degradative fluorescent fragment (retention time 15.7 min) was integrated and the peak area was found. The inhibitory activity of the sample was calculated using the following equation.

Inhibition (%)=100−[(Peak area from sample group)/(Peak area from control group)×100]

TABLE 1
β-Secretase inhibitory activities of sesamolin in different ratios
from S. indicum (seed).

| | Concentration | | Inhibition |
|---|---|---|---|
| Samples | μg/ml | Peak area ± S.D. | (%) |
| Control | — | 4284231 ± 33852 | — |
| Sesame extract with | 50 | 3928639 ± 34153 | 8.3 |
| 20% Sesamolin | | | |
| Sesame extract with | 50 | 3250017 ± 41345 | 24.14 |
| 40% Sesamolin | | | |
| Sesame extract with | 50 | 3133486 ± 23481 | 26.86 |
| 55% Sesamolin | | | |
| Sesame extract with | 50 | 3044802 ± 29634 | 28.93 |
| 60% Sesamolin | | | |
| Sesamolin 80% | 50 | 2448009 ± 48324 | 42.86 |
| Inhibitor | 2 | 2256512 ± 24323** | 47.33 |

Each value represents the mean ± S.D. of triplicates.
Significantly different from the control at **P < 0.01.

Inhibitor: Lys-Thr-Glu-Glu-Ile-Ser-Glu-Val-Asn-Sta-Val-Ala-Glu-Phe.

The result showed inhibitory assay against beta-secretase in all sample groups, and the activity is increasing as the concentration of sesamolin is increased. Enriched 80% sesamolin showed the most potent inhibitory activity (42.86%) among the extract tested. This indicates that 80% sesamolin will show 5 times better beta-secretase inhibitor activity than 20% sesamolin.

### Example 18

β-Secretase Inhibitory Activity of Essential Oil of Turmeric (EOT) Having Different Percentages of α-Turmerone.

Material Used

β-Secretase (human, recombinant). A fluorescent substrate [MOCAc-Ser-Glu-Val-Asn-Leu-Asp-Ala-Glu-Phe-Arg-Lys (Dnp) Arg-Arg-NH2] and a β-secretase inhibitor[Lys-Thr-Glu-Glu-Ile-Ser-Glu-Val-Asn-Sta-Val-Ala-Glu-Phe(Sta:(3S,4S)-4-amino-3-hydroxy-6-methylheptanoic acid)]. Other reagents used in this study were analytical grade. Essential oil of turmeric (EOT) with different percentage of α-turmerone prepared as per examples 2.

Procedure

Samples in a dimethylsulfoxide solution (2 μl), at an appropriate concentration, were diluted with 78 μl of assay buffer (20 mM acetate buffer, pH=4.5 containing 1% triton X-100) in a 96-well microplate. 10 μl of β-secretase solution in assay buffer (17.4 μg protein/ml) was added to the diluted solution, and incubated at 37° C. for 20 min. After incubation, 10 μl of substrate solution with assay buffer (0.1 mM) was added and incubated at 37° C. for 2 h. Fifty μl of reaction solution was then added to 450 μl of 2.5 M sodium acetate solution to terminate the reaction. The diluted solution was analyzed by HPLC applying the conditions listed below. Column: L-column ODS (Chemicals Evaluation and Research Institute, Tokyo, Japan, 4.6 i.d.×250 mm), mobile phase; 0.1% (v/v) formic acid/acetonitrile (0.1% (v/v) formic acid); 0 min (9:1), 17.5 min (5.5:4.5), 17.6 min (0.5:9.5), 22.5 min (0.5:9.5), column temperature; 40° C., flow rate; 1 ml/min, detection; fluorescent of excitation at 325 nm and emission at 395 nm, injection volume; 20 μl. The peak area of the peak derived from the degradative fluorescent fragment (retention time 15.7 min) was integrated and the peak area was found. The inhibitory activity of the sample was calculated using the following equation.

Inhibition (%)=100−[(Peak area from sample group)/(Peak area from control group)×100]

TABLE 1
β-Secretase inhibitory activities of essential oil of turmeric with different percentage
of alpha turmerone.

| | | | Inhibition |
|---|---|---|---|
| Samples | Concentration μg/ml | Peak area ± S.D. | (%) |
| Control | — | 4391224 ± 35462 | 0 |
| Essential oil of turmeric with 30% α- | 50 | 4075055 ± 31437 | 7.2 |
| turmerone | | | |
| Essential oil of turmeric enriched | 50 | 3231940 ± 25349 | 26.4 |
| with 40% α-turmerone | | | |
| Essential oil of turmeric enriched | 50 | 2704993 ± 29537 | 38.4 |
| with 60% α-turmerone | | | |
| Essential oil of turmeric enriched | 50 | 2432738 ± 31475 | 44.6 |
| with 80% α-turmerone | | | |
| Inhibitor | 2 | 2386115 ± 25498** | 45.67 |

Each value represents the mean ± S.D. of triplicates.
Significantly different from the control at **P < 0.01.

Inhibitor: Lys-Thr-Glu-Glu-Ile-Ser-Glu-Val-Asn-Sta-Val-Ala-Glu-Phe.

From the result it was clear that essential oil of turmeric enriched with α-turmerone showed inhibitory assay against beta-secretase. Essential oil of turmeric enriched with 80% α-turmerone showed 44.6% inhibition, which is 6 times higher inhibition than essential oil of turmeric with 30% α-turmerone. Essential oil of turmeric enriched with 60% α-turmerone showed 38.4% inhibition compared to essential oil of turmeric with 30% and 40% α-turmerone. This indicates that essential oil of turmeric enriched with α-turmerone showed beta-secretase inhibitor activity.

### Example 19

β-Secretase Inhibitory Activity of a Combination of Essential Oil of Turmeric (EOT) Enriched with α-Turmerone and Further Including Different Ratios of Sesamolin.

Material Used

β-Secretase (human, recombinant), A fluorescent substrate [MOCAc-Ser-Glu-Val-Asn-Leu-Asp-Ala-Glu-Phe-Arg-Lys (Dnp) Arg-Arg-NH2] and a β-secretase inhibitor[Lys-Thr-Glu-Glu-Ile-Ser-Glu-Val-Asn-Sta-Val-Ala-Glu-Phe(Sta:(3S,4S)-4-amino-3-hydroxy-6-methylheptanoic acid)]. Other reagents used in this study were analytical grade. Combination of essential oil of turmeric enriched with α-turmerone and sesamin-sesamolin in different ratios prepared as per examples 11-15.

Procedure

Samples in a dimethylsulfoxide solution (2 μl), at an appropriate concentration, were diluted with 781 of assay buffer (20 mM acetate buffer, pH=4.5 containing 1% triton X-100) in a 96-well microplate. 10 μl of β-secretase solution in assay buffer (17.4 μg protein/ml) was added to the diluted solution, and incubated at 37° C. for 20 min. After incubation, 100l of substrate solution with assay buffer (0.1 mM) was added and incubated at 37′C for 2 h. Fifty μl of reaction solution was then added to 450 μl of 2.5 M sodium acetate solution to terminate the reaction. The diluted solution was analyzed by HPLC applying the conditions listed below. Column: L-column ODS (Chemicals Evaluation and Research Institute, Tokyo, Japan, 4.6 i.d.×250 mm), mobile phase: 0.1% (v/v) formic acid/acetonitrile (0.1% (v/v) formic acid): 0 min (9:1), 17.5 min (5.5:4.5), 17.6 min (0.5:9.5), 22.5 min (0.5:9.5), column temperature; 40° C. flow rate; 1 m/min, detection: fluorescent of excitation at 325 nm and emission at 395 nm, injection volume: 20 μl. The peak area of the peak derived from the degradative fluorescent fragment (retention time 15.7 min) was integrated and the peak area was found. The inhibitory activity of the sample was calculated using the following equation.

Inhibition (%)=100−[(Peak area from sample group)/(Peak area from control group)×100]

TABLE 1
β-Secretase inhibitory activities of combination of essential oil of turmeric enriched
with α-turmerone and sesamolin in different ratios.

| | Concentration | | Inhibition |
|---|---|---|---|
| Samples | μg/ml | Peak area ± S.D. | (%) |
| Control | — | 4678456 ± 40218 | — |
| EOT with 30% α-turmerone + 20% | 50 | 3546269 ± 31427 | 24.2 |
| sesamolin in 8:1 ratio | | | |
| EOT with 30% α-turmerone + 40% | 50 | 3457378 ± 35462 | 26.1 |
| sesamolin in 8:1 ratio | | | |
| EOT with 60% α-turmerone + 40% | 50 | 3012925 ± 41846 | 35.6 |
| sesamolin in 8:1 ratio | | | |
| EOT with 30% α-turmerone + 40% | 50 | 3298311 ± 34864 | 29.5 |
| sesamolin in 5:1 ratio | | | |
| EOT with 30% α-turmerone + 80% | 50 | 2180160 ± 41764 | 53.4 |
| sesamolin in 8:1 ratio | | | |
| Inhibitor | 2 | 2418426 ± 23621** | 48.31 |

Each value represents the mean ± S.D. of triplicates.
Significantly different from the control at **P < 0.01.

Inhibitor: Lys-Thr-Glu-Glu-Ile-Ser-Glu-Val-Asn-Sta-Val-Ala-Glu-Phe.

Result shows that combination of essential oil of turmeric with 30% α-turmerone and sesame seed extract with 80% sesamolin in 8:1 ratio significantly higher beta-secretase inhibitory activity (53.4%) compared to combination of essential oil of turmeric with 30% α-turmerone and sesame seed extract with 40% sesamolin in 8:1 ratio (26.1% inhibition). EOT with 30% α-turmerone and 20% sesamolin in 8:1 ratio showed 24.2% inhibition. All other groups with different ratio of α-turmerone and different ratios of sesamolin also showed inhibition.

### Example 20

β-Secretase Inhibitory Activities of Curcuminoid Blended with Essential Oil of Turmeric Enriched with Different Percentage of α-Turmerone in 10:1 and 12:1 Ratios

Material Used

β-Secretase (human, recombinant), A fluorescent substrate [MOCAc-Ser-Glu-Val-Asn-Leu-Asp-Ala-Glu-Phe-Arg-Lys (Dnp) Arg-Arg-NH2] and a β-secretase inhibitor[Lys-Thr-Glu-Glu-Ile-Ser-Glu-Val-Asn-Sta-Val-Ala-Glu-Phe(Sta:(3S,4S)-4-amino-3-hydroxy-6-methylheptanoic acid)]. Other reagents used in this study were analytical grade. Combination of curcuminoids and essential oil of turmeric enriched with α-turmerone prepared as per examples 3-9.

Procedure

Samples in a dimethylsulfoxide solution (2 μl), at an appropriate concentration, were diluted with 78 μl of assay buffer (20 mM acetate buffer, pH=4.5 containing 1% triton X-100) in a 96-well microplate. 10 μl of β-secretase solution in assay buffer (17.4 μg protein/ml) was added to the diluted solution, and incubated at 37° C. for 20 min. After incubation, 10 μl of substrate solution with assay buffer (0.1 mM) was added and incubated at 37° C. for 2 h. Fifty μl of reaction solution was then added to 450 μl of 2.5 M sodium acetate solution to terminate the reaction. The diluted solution was analyzed by HPLC applying the conditions listed below. Column; L-column ODS (Chemicals Evaluation and Research Institute, Tokyo, Japan, 4.6 i.d.×250 mm), mobile phase: 0.1% (v/v) formic acid/acetonitrile (0.1% (v/v) formic acid): 0 min (9:1), 17.5 min (5.5:4.5), 17.6 min (0.5:9.5), 22.5 min (0.5:9.5), column temperature; 40° C. flow rate; 1 ml/min, detection: fluorescent of excitation at 325 nm and emission at 395 nm, injection volume, 20 μl. The peak area of the peak derived from the degradative fluorescent fragment (retention time 15.7 min) was integrated and the peak area was found. The inhibitory activity of the sample was calculated using the following equation.

Inhibition (%)=100−[(Peak area from sample group)/(Peak area from control group)×100]

TABLE 1
β-Secretase inhibitory activities of combination of curcuminoids and essential oil of
turmeric enriched with α-turmerone in 10:1 to 12:1 ratios.

| | Concentration | | |
|---|---|---|---|
| Samples | μg/ml | Peak area ± S.D. | Inhibition (%) |
| Control | — | 4490423 ± 39845 | 0 |
| curcuminoid alone | 50 | 4090775 ± 31278 | 8.9 |
| Curcuminoids + EOT with 80% α- | 50 | 2429318 ± 41264 | 45.9 |
| turmerone in 12:1 ratio | | | |
| Curcuminoids + EOT with 60% α- | 50 | 2766100 ± 35413 | 38.4 |
| turmerone in 12:1 ratio | | | |
| Curcuminoids + EOT with 40% α- | 50 | 3179219 ± 29476 | 29.2 |
| turmerone in 12:1 ratio | | | |
| Curcuminoids + EOT with 30% α- | 50 | 3524982 ± 34721 | 21.5 |
| turmerone in 12:1 ratio | | | |
| Curcuminoids + EOT with 30% α- | 50 | 3709289 ± 38426 | 17.4 |
| turmerone in 10:1 ratio | | | |
| Inhibitor | 2 | 2299129 ± 25842** | 48.8 |

Each value represents the mean ± S.D. of triplicates.
Significantly different from the control at **P < 0.01.

Inhibitor: Lys-Thr-Glu-Glu-Ile-Ser-Glu-Val-Asn-Sta-Val-Ala-Glu-Phe.

Result showed combination of curcuminoids and essential oil of turmeric enriched with 80% α-turmerone in 12:1 ratio showed higher beta-secretase inhibitory activity (45.9%) compared curcuminoid alone (8.9% inhibition). That is 5 percent high inhibition. Curcuminoids and essential oil of turmeric enriched with 60% α-turmerone in 12:1 ratios showed 38.4% inhibition. It is clear that beta secreatase inhibitory activity of curcuminoids is enhanced by adding essential oil of turmeric enriched with alpha turmerone.

### Example 21

Synergistic effect of different combination of actives by comparing experimentally observed inhibition and expected inhibition based on stoichiometric amounts of extracts is provided below.

TABLE 1
Synergistic effect of different combination of actives by comparing
experimentally observed inhibition and expected inhibition based
on stoichiometric amounts of extracts.

| | | | Predicted | |
|---|---|---|---|---|
| | | | inhibition | Experimentally |
| | | | based on | observed Inhibition of |
| | Group | Dose | stoichiometry | β-Secretase (%) |
| Alone | Curcuminoid alone | 50 μg/ml | | 8.9 |
| | EOT with 80% α-t | 50 μg/ml | | 44.6 |
| | EOT with 30% α-t | 50 μg/ml | | 7.2 |
| | Sesamolin 80% | 50 μg/ml | | 42.46 |
| Combination | Curcuminoids with | 50 μg/ml | 11.65 | 45.9 |
| | EOT with 80% α-t | | | |
| | in 12:1 ratio | | | |
| | EOT with 30% α-t | 50 μg/ml | 11.1 | 53.4 |
| | with Sesamolin | | | |
| | 80% in 8:1 ratio | | | |

In vitro study was done with curcuminoids showed slight inhibition of 3-Secretase. Better results were observed with essential oil of turmeric which was enriched with α-turmerone. In vitro study done with essential oil of turmeric with 80% purity of α-turmerone showed clinically significant result, an inhibition of about 44.6% (From example 18, table 1). The in vitro study conducted using sesame seed extract, particularly one containing sesamolin, also shown inhibition of β-Secretase. An extract with 80% purity of sesamolin showed β-secretase inhibition up to 42.86% (from Example 17, table 1).

The combination of curcuminoids with essential oil of turmeric with 80% purity of α-turmerone is in 12:1 ratio was provided at a dosage of 50 μg/ml. In the composition 12 out of 13 parts of 50 μg/ml was curcuminoids, which corresponds to 46.15 μg/ml curcuminoids. The composition had 3.85 μg/ml essential oil of turmeric with 80% purity of α-turmerone. Inhibition was directly proportional to the dosage administered. Inhibition based on stoichiometric amounts of the extracts was calculated. Inhibition based on 46.15 μg/ml curcuminoids in the composition of curcuminoids and essential oil was calculated to be about 8.2%. Inhibition based on 3.85 μg/ml essential oil of turmeric (80% α-turmerone) in the composition of curcuminoids and essential oil was calculated to be about 3.45%. Therefore, the composition having curcuminoids and essential oil was calculated or predicted to inhibit to a total of about 11.65% based on the stoichiometric amounts of curcuminoids and essential oil with α-turmerone in the composition. However, as the in vitro study above showed, composition having curcuminoids and essential oil of turmeric (80% α-turmerone) in 12:1 ratio at 50 μg/ml inhibited β-Secretase about 45.9% (Example 20, table 1). Therefore, the actual inhibition was almost four times higher than the inhibition predicted based on stoichiometric amounts of the curcuminoid and essential oil extracts in the composition. Therefore, the composition having curcuminoids and essential oil of turmeric (80% α-turmerone) exhibited a synergetic effect for the inhibition of β-secretase.

A combination of essential oil of turmeric (30% α-turmerone) with sesame seed extract (80% sesamolin) in 8:1 ratio was administered at a dosage of 50 μg/ml. In the composition, 8 out of 9 parts of 50 μg/ml was essential oil of turmeric (having 30% α-turmerone), which corresponded to 44.45 μg/ml essential oil of turmeric (30% α-turmerone) and 5.55 μg/ml was sesame seed extract (having 80% sesamolin). The inhibition was found to be directly proportional to the dosage. Since 50 μg/ml of essential oil of turmeric (30% α-turmerone) showed an inhibition of 7.2% (from example 18, table 1), the inhibition at 44.45 g/ml was calculated to be 6.4% [44.45*7.2/50=6.4]. At 50 μg/ml of sesame seed extract (80% sesamolin) showed an inhibition of 42.46% (from Example 17, table 1), therefore, inhibition at 5.55 μg/ml was calculated to be about 4.7% [5.55*42.46/50=4.7]. So the predicted inhibition calculated based on stoichiometric amounts of essential oil extract of turmeric and sesame oil extract in the composition having 44.45 μg/ml essential oil of turmeric (30% α-turmerone) and 5.55 μg/ml sesame seed extract (80% sesamolin) was 6.4+4.7, which is about 11.1%.

However, the in vitro study showed that the combination of essential oil of turmeric with 30% α-turmerone and sesame seed extract with 80% sesamolin in 8:1 ratio at 50 μg/ml inhibited β-Secretase about 53.4% (from example 19, table 1). Therefore, the actual inhibition was almost five times the calculated or predicted inhibition of 11.1%. Therefore, the composition having essential oil of turmeric and sesame seed extract exhibits a synergetic effect on the inhibition of β-Secretase.

### Example 22

β-Secretase Inhibitory Activities of Combination of Curcuminoids and 8:1 Ratio of Essential Oil of Turmeric (EOT) Enriched with 30% α-Turmerone and 40% Sesamolin in 70:30 Ratio.

Material Used

β-Secretase (human, recombinant), A fluorescent substrate [MOCAc-Ser-Glu-Val-Asn-Leu-Asp-Ala-Glu-Phe-Arg-Lys (Dnp) Arg-Arg-NH2] and a β-secretase inhibitor[Lys-Thr-Glu-Glu-Ile-Ser-Glu-Val-Asn-Sta-Val-Ala-Glu-Phe(Sta:(3S,4S)-4-amino-3-hydroxy-6-methylheptanoic acid)]. Other reagents used in this study were analytical grade. Combination curcuminoids and 8:1 ratio of essential oil of turmeric (EOT) enriched with 30% α-turmerone and 40% sesamolin in 70:30 ratio prepared as per example 20.

Procedure

Samples in a dimethylsulfoxide solution (2 μl), at an appropriate concentration, were diluted with 78 μl of assay buffer (20 mM acetate buffer, pH=4.5 containing 1% triton X-100) in a 96-well microplate. 10 μl of β-secretase solution in assay buffer (17.4 μg protein/ml) was added to the diluted solution, and incubated at 37° C. for 20 min. After incubation, 10 μl of substrate solution with assay buffer (0.1 mM) was added and incubated at 37° C. for 2 h. 50 μl of reaction solution was then added to 450 μl of 2.5 M sodium acetate solution to terminate the reaction. The diluted solution was analyzed by HPLC applying the conditions listed below. Column: L-column ODS (Chemicals Evaluation and Research Institute, Tokyo, Japan, 4.6 i.d.×250 mm), mobile phase; 0.1% (v/v) formic acid/acetonitrile (0.1% (v/v) formic acid); 0 min (9:1), 17.5 min (5.5:4.5), 17.6 min (0.5:9.5), 22.5 min (0.5:9.5), column temperature; 40° C., flow rate; 1 ml/min, detection; fluorescent of excitation at 325 nm and emission at 395 nm, injection volume; 20 μl. The peak area of the peak derived from the degradative fluorescent fragment (retention time 15.7 min) was integrated and the peak area was found. The inhibitory activity of the sample was calculated using the following equation.

Inhibition (%)=100−[(Peak area from sample group)/(Peak area from control group)×100]

TABLE 1
β-Secretase inhibitory activities of curcuminoids and 8:1 ratio of
essential oil of turmeric (EOT) enriched with 30% α-turmerone
and 40% sesamolin in 70:30 ratio.

| | Concentration | Peak area ± | Inhibition |
|---|---|---|---|
| Samples | μg/ml | S.D. | (%) |
| Control | — | 4398756 ± 37493 | 0 |
| Curcuminoids + EOT with | 50 | 2260960 ± 45875 | 48.6 |
| 30% α-turmerone + 40% | | | |
| sesamolin in 70:30 ratio | | | |
| Curcuminoids alone | 50 | 3980874 ± 23451 | 9.5 |
| EOT with 30% α-turmerone + | 50 | 3193496 ± 28426 | 27.4 |
| 40% sesamolin in 8:1 ratio | | | |
| Inhibitor | 2 | 2385632 ± 27453** | 45.77 |

Each value represents the mean ± S.D. of triplicates.
Significantly different from the control at **P < 0.01.

Inhibitor: Lys-Thr-Glu-Glu-Ile-Ser-Glu-Val-Asn-Sta-Val-Ala-Glu-Phe.

From the study it was clear that combination of curcuminoids and EOT with 30% α-turmerone and 40% sesamolin in 8:1 ratio showed 5 times higher beta-secretase inhibitory activity (48.6%) compared to curcuminoid alone (9.5%).

### Example 23

Study for Finding the Presence of α-Turmerone, β-Turmerone, Ar-Turmerone, Sesamin and Sesamolin in Brain and Serum of Mice by Administering a Combination of Purified Essential Oil of Turmeric and Sesame Seed Extract in 5:1 Ratio.

Five week old mice (Shimizu Laboratory Supplies, Kyoto, Japan) were used for the study. The mice were kept in a humidity-controlled room under normal and uniform conditions like 12 hours light and dark cycle and at 25±1° C. Water and standard diet (Oriental Yeast. Tokyo, Japan) were given ad-libitum. After habituation for 1 week, sample was orally administered. The animals were divided as shown in table 1.

| | |
|---|---|
| Group 1 | Vehicle |
| Group 2 | Essential oil of turmeric with 40% α-turmerone:sesame seed |
| | extract with 25% sesamolin in 5:1 ratio. |
| Group 3 | Essential oil of turmeric with 60% α-turmerone:sesame seed |
| | extract with 25% sesamolin in 5:1 ratio. |
| Group 4 | Essential oil of turmeric with 80% α-turmerone:sesame seed |
| | extract with 25% sesamolin in 5:1 ratio. |
| Group 5 | Essential oil of turmeric with 40% α-turmerone:sesame seed |
| | extract with 80% sesamolin in 5:1 ratio. |
| Group 6 | Sesame seed extract with 25% sesamolin. |
| Group 7 | Essential oil of turmeric with 40% α-turmerone. |

After 30 min, blood and brain were collected. Serum was obtained by centrifugation at 3,000 rpm for 10 min. Brain samples were quickly frozen in liquid nitrogen, and stored at −80° C. until analysis by LC-MS.

LC-MS Analysis of Sesamin and Sesamolin in Serum and Tissue Sample.

Plasma extraction: Plasma sample (100 μl) and 500 μl of MeCN were combined and extracted by 3 min of vortex. The solution was then centrifuged (3,000 rpm for 3 min). The supernatant was evaporated under reduced pressure and the residue was dissolved into 5051l of MeCN for LC-MS analysis.

Brain extraction: Brain sample (30 mg) was pulverized and extracted with 150 μl of MeCN (methyl cyanide or acetonitrile). After 10 min of vortex and sonication, the suspension was centrifuged at 3,000 rpm for 3 min. The supernatant was evaporated under reduced pressure and the residue was dissolved into 755l of MeCN for LC-MS analysis.

Sesamin analysis: UPLC-MS system (LCMS-8050, Shiamdzu. Kyoto, Japan, equipped with Lab solutions ver. 5.75) was used. Mobile phase used is methanol (MeOH)/5 mM ammonium acetate in water (4:1), retention time: 2.25 min, injection volume: 1 μl.

Sesamolin analysis: UPLC-MS system (LCMS-8050, Shiamdzu, Kyoto, Japan, equipped with Lab solutions ver. 5.75) was used. Mobile phase used is MeOH/5 mM ammonium acetate in water (4:1) to (1:0) at 2.5 min with linear gradient and back to (4:1) at 2.51 min, retention time: 2.55 min.

GC-MS Analysis of α-Turmerone, β-Turmerone, Ar-Turmerone Analysis:

Plasma extraction: Plasma was weighed and loaded on a SPE catridge previously activated with 1.5 ml of 0.05% acetic acid in methanol and washed with 1.5 ml of 0.05% acetic acid in water. After sample loading, the SPE tubes (Strata-X 33p polymeric reversed phase: 8B-S100-UBJ phenomenex) were washed with 1.5 ml of 0.05% acetic acid in water and the analytes eluted with 4×1 ml of methanol in a test tube. The organic phase was evaporated to dryness using high performance personal evaporating system (Genevac UK).

Turmerone analysis: A GC system (Shimadzu Corporation, Kyoto, Japan) consisting of a GCMS-QP 2010 ultra Gas Chromatograph mass spectrometer and an electron ionization mode was used. The samples were separated on Rxi-5Sil MS (30 m, 0.25 mmID) column. The plasma sample was reconstituted with 1 ml of hexane and transferred into a micro-vial. A 1 μl aliquot was injected in GC-MS system & analyzed the alpha, beta & Ar-turmerone. Data acquisition and quantitation were performed using Shimadzu GCMS solution software.

TABLE 2
Sesamin, Sesamolin, α-Turmerone, β-turmerone and Ar-turmerone in the serum

| | | Sesamin | Sesamolin | α-Turmerone | β-turmerone | Ar-turmerone |
|---|---|---|---|---|---|---|
| Group | Dosage | (ng/ml) | (ng/ml) | (ng/ml) | (ng/ml) | (ng/ml) |
| Group 1 | | 0 | 0 | 0 | 0 | 0 |
| Group 2 | 25 mg/Kg | 15.9 | 38.9 | 26.2 | 10.77 | 6.89 |
| Group 3 | 25 mg/Kg | 22.3 | 45.7 | 30.5 | 9.4 | 5.14 |
| Group 4 | 25 mg/Kg | 30.5 | 53.1 | 42.3 | 6.1 | 2.5 |
| Group 5 | 25 mg/Kg | — | 62.4 | 29.3 | 10.4 | 5.3 |
| Group 6 | 25 mg/Kg | 5.2 | 9.8 | — | — | — |
| Group 7 | 25 mg/Kg | — | — | 12.9 | 4.98 | 2.63 |

TABLE 3
Sesamin, Sesamolin, α-Turmerone, β-turmerone and Ar-turmerone in the brain

| | | Sesamin | Sesamolin | α-Turmerone | β-turmerone | Ar-turmerone |
|---|---|---|---|---|---|---|
| Group | Dosage | (ng/mg) | (ng/mg) | (ng/mg) | (ng/mg) | (ng/mg) |
| Group 1 | | 0 | 0 | 0 | 0 | 0 |
| Group 2 | 25 mg/Kg | 25.62 | 50.19 | 19.8 | 8.6 | 4.8 |
| Group 3 | 25 mg/Kg | 30.5 | 62.3 | 29.6 | 7.9 | 3.1 |
| Group 4 | 25 mg/Kg | 34.8 | 71.7 | 41.2 | 5.4 | 1.2 |
| Group 5 | 25 mg/Kg | — | 85.6 | 21.3 | 8.9 | 5.1 |
| Group 6 | 25 mg/Kg | 8.5 | 12.5 | — | — | — |
| Group 7 | 25 mg/Kg | — | — | 9.5 | 4.5 | 2.1 |

Result showed that after administering essential oil of turmeric with 40% α-turmerone:sesame seed extract with 25% sesamolin in 5:1 ratio in mice showed the availability of sesamin, sesamolin, α-turmerone, β-turmerone and Ar-turmerone in the brain and serum. These results suggest that these active compounds had moved to the brain through blood brain barrier and can inhibit β-secretase.

### Example 24

Study for Finding the Bioavailability of Curcumin, Demethoxycurcumin and Bisdemethoxycurcumin in Plasma of Rat by Administering a Combination of Curcuminoid and Essential Oil of Turmeric Enriched with α-Turmerone in 12:1 Ratio.

Albino rats weighing 150-200 gm of both sexes were used for the study. The rats were kept individually in polypropylene cages and maintained in well ventilated room under normal and uniform conditions like 12 hours light and dark cycle and at 26+2° C. Water and feed were given ad-libitum. The animals were divided into 4 groups and 3 animals were used for each group. Dosage is 60 mg/Kg. Animals were divided as shown in table 1.

| | |
|---|---|
| Group 1 | Vehicle |
| Group 2 | curcuminoids + essential oil of turmeric enriched with 60% α- |
| | turmerone in 12:1 ratio. |
| Group 3 | curcuminoids + essential oil of turmeric enriched with 45% Ar- |
| | tumerone in 12:1 ratio. |
| Group 4 | curcuminoids alone. |

Each sample was orally administered. After 2 hrs, blood was collected. Plasma was obtained by centrifugation at 3,000 rpm for 10 min and curcumin, demethoxy curcumin and bisdemethoxycurcumin was analysed by LC-MS.

LC-MS Analysis of Curcuminoids in Plasma Sample.

Plasma Extraction

Plasma was weighed and loaded on a SPE catridge previously activated with 1.5 ml of 0.05% acetic acid in methanol and washed with 1.5 ml of 0.05% acetic acid in water. After sample loading, the SPE tubes (Strata-X 33μ polymeric reversed phase; 8B-S100-UBJ phenomenex) were washed with 1.5 ml of 0.05% acetic acid in water and the analytes eluted with 4×1 ml of methanol:acetonitrile (1:1) in a test tube. The organic phase was evaporated to dryness using high performance personal evaporating system (Genevac UK).

Curcuminoids Analysis:

An LC system (Waters, Corporation, Milford, U.S.A) consisting of an Acquity ultra Performance LC and electrospray chemical ionization tandem mass spectrometer (ESCi-MS/MS; Waters) was used. The samples were separated on Ambient BEH C18 (2.1×50 mm), 1.7μ or Equivalent L1 column. The calibration curves of bisdemethoxy curcumin, demethoxy curcumin and curcumin were linear over the concentration range of 1-800 ppb. The LOQ for curcumin, demethoxy curcumin and bisdemethoxy curcumin were 10.0 ppb. The plasma sample was reconstituted with 1 ml of acetonitrile: water containing 0.1% Formic acid (1:1) and transferred into a micro-vial. A 5 μl aliquot was injected in LC-MS/MS system & analyzed the curcuminoids & metabolites. Data acquisition and quantitation were performed using MassLynx software version 4.1.

TABLE 2
Curcuminoids availability in the plasma

| Groups | Curcumin (ng/g) | DMC (ng/g) | BDMC (ng/g) |
|---|---|---|---|
| Group 1 | 0 | 0 | 0 |
| Group 2 | 38.8 | 24.87 | 14.7 |
| Group 3 | 8.9 | ND | ND |
| Group 4 | 1.99 | ND | ND |

The results as shown in Table 2 indicate that curcumin, demethoxycurcumin and bisdemethoxycurcumin were detected in group 2 animals fed with curcuminoids and essential oil of turmeric enriched with 60% α-turmerone in 12:1 ratio. Animals fed with curcuminoids and essential oil of turmeric enriched with 45% Ar-tumerone in 12:1 ratio (Group 3) showed detection of curcumin only in plasma. Demethoxycurcumin and bisdemethoxy curcumin was not detected in rats fed with curcuminoids and essential oil of turmeric enriched with 45% Ar-tumerone in 12:1 ratio.

Animals fed with curcuminoid mixture alone (Group 4) showed low detection of curcumin in plasma.

### Example 25

Study for Finding the Bioavailability of Curcumin, Demethoxycurcumin and Bisdemethoxycurcumin in Brain of Rat by Administering a Combination of Curcuminoid and Essential Oil of Turmeric Enriched with α-Turmerone in 12:1 Ratio.

Albino rats weighing 200-250 gm of both sexes were used for the study. The rats were kept individually in polypropylene cages and maintained in well ventilated room under normal and uniform conditions of 12 hours light and dark cycle and at 26+2° C. Water and feed were given ad-libitum. The animals were divided into 4 groups of 3 animals in each group having an oral dosage of 60 mg/Kg. Animals were divided as shown in table 1.

| | |
|---|---|
| Group 1 | Vehicle |
| Group 2 | curcuminoids + essential oil of turmeric enriched with 60% α- |
| | turmerone in 12:1 ratio. |
| Group 3 | curcuminoids + essential oil of turmeric enriched with 45% Ar- |
| | tumerone in 12:1 ratio. |
| Group 4 | curcuminoids alone. |

After 24 hours, rats were killed under ether anaesthesia, and brain was collected. Brain samples were quickly frozen in liquid nitrogen, and stored at −80° C. until analysis by LC-MS.

LC-MS Analysis of Curcuminoids in Brain Sample.

Brain extraction: Brain sample (30 mg) was pulverized and extracted with 2 ml ethyl acetate. Ethyl acetate fraction was collected and repeated the extraction two more times with ethyl acetate. The entire ethyl acetate fraction was combined. After 10 min of vortex and sonication, the suspension was centrifuged at 3,000 rpm for 3 min. The supernatant was evaporated under reduced pressure to form a residue for LC-MS analysis.

Curcuminoids Analysis:

An LC system (Waters, Corporation, Milford, U.S.A) consisting of an Acquity ultra Performance LC and electrospray chemical ionization tandem mass spectrometer (ESCi-MS/MS; Waters) was used. The samples were separated on Ambient BEH C18 (2.1×50 mm), 1.7 g or Equivalent L1 column. The calibration curves of bisdemethoxy curcumin, demethoxy curcumin and curcumin were linear over the concentration range of 1-800 ppb. The LOQ for curcumin, demethoxy curcumin and bisdemethoxy curcumin were 10.0 ppb. The residue was reconstituted with 1 ml of acetonitrile: water containing 0.1% Formic acid (1:1) and transferred into a micro-vial. A 5 μl aliquot was injected in LC-MS/MS system & analyzed the curcuminoids. Data acquisition and quantitation were performed using MassLynx software version 4.1.

GC-MS Analysis of α-Turmerone, β-Turmerone, Ar-Turmerone Analysis:

Turmerone analysis: A GC system (Shimadzu Corporation. Kyoto, Japan) consisting of a GCMS-QP 2010 ultra Gas Chromatograph mass spectrometer and an electron ionization mode was used. The samples were separated on Rxi-5Sil MS (30 m, 0.25 mmID) column. The residue was reconstituted with 1 ml of hexane and transferred into a micro-vial. A 1 μl aliquot was injected in GC-MS system & analyzed the alpha, beta & Ar-turmerone. Data acquisition and quantitation were performed using Shimadzu GCMS solution software.

TABLE 2
Curcumin, demethoxycurcumin (DMC), bisdemethoxycurcumin (BDMC),
α-turmerone, β-turmerone and Ar-turmerone in the brain.

| | Curcumin | DMC | BDMC | α-turmerone | β-turmerone | Ar-turmerone |
|---|---|---|---|---|---|---|
| Group | (ng/mg) | (ng/mg) | (ng/mg) | (ng/mg) | (ng/mg) | (ng/mg) |
| Group 1 | 0 | 0 | 0 | 0 | 0 | 0 |
| Group 2 | 1050.65 | 124.33 | 15.17 | 38.6 | 6.1 | 2.3 |
| Group 3 | 256.3 | 18.95 | 0 | 3.4 | 0 | 21.4 |
| Group 4 | 2.3 | 0 | 0 | 0 | 0 | 0 |

The results as shown in Table 2 indicate that curcumin, demethoxycurcumin and bisdemethoxy curcumin was detected in brain of group 2 animals fed with curcuminoids and essential oil of turmeric enriched with 60% α-turmerone in 12:1 ratio. α-turmerone, 0-turmerone and Ar-turmerone was also detected in brain of group 2 animals fed with curcuminoids and essential oil of turmeric enriched with 60% α-turmerone in 12:1 ratio. Animals fed with curcuminoids and essential oil of turmeric enriched with 45% Ar-tumerone in 12:1 ratio (Group 3) showed small detection of curcumin, demethoxycurcumin, α-Turmerone and Ar-turmerone in brain. Demethoxycurcumin and bisdemethoxy curcumin was not detected in brain of rats fed with curcuminoids alone but a slight detection of curcumin was only found.

We have brought out the novel features of the invention by explaining some of the preferred embodiments under the invention, enabling any person skilled in the art to understand and visualize our invention. It is also to be understood that the invention is not limited in its application to the details set forth in the above description or illustrated in the drawings. Although the invention has been described in considerable detail with particular reference to certain preferred embodiments thereof, variations and modifications can be effected within the spirit and scope of the invention as described herein above and as defined in the appended claims.

I claim:

1. A composition for inhibiting beta secretase activity,
wherein the composition comprises a curcuminoid mixture and an essential oil of turmeric,
wherein the curcuminoid mixture comprises curcumin, demethoxycurcumin, and, bisdemethoxycurcumin,
wherein the essential oil of turmeric comprises about 30% to about 80% of α-turmerone,
wherein a weight ratio of the curcuminoid mixture to the essential oil of turmeric ranges from about 1:50 to about 99:1.

2. The composition of claim 1 further comprising enhancing bioavailability of a constituent, wherein the constituent is selected from the group consisting of curcumin, demethoxycurcumin, bisdemethoxycurcumin, α-turmerone, β-turmerone, and ar-turmerone.

3. A dosage form comprising the composition of claim 1, the dosage form selected from the group consisting of capsule, tablet, granule, sachet, powder, paste, infusion, ampoule, solution, suspension, emulsion, pills, and, cream.

4. A method for inhibiting beta secretase in brain, the method comprising administering the composition of claim 1.

5. A method of treating a central nervous system disorder, the method comprising administering the composition of claim 1, the central nervous system disorder selected from the group consisting of epilepsy, migraine, Huntington's disease, Alzheimer's disease, depression, Parkinson's disease, Tourette syndrome, dystonia, multiple sclerosis, meningitis, lupus, fibromyalgia, and, bipolar disorder.

6. A method of enhancing bioavailability of a constituent in brain, the method comprising administering the composition of claim 1, the constituent selected from the group consisting of curcumin, demethoxycurcumin, bisdemethoxycurcumin, α-turmerone, β-turmerone, and, ar-turmerone.

7. A method of enhancing bioavailability of a constituent in serum, the method comprising administering the composition of claim 1, the constituent selected from the group consisting of curcumin, demethoxycurcumin, bisdemethoxycurcumin, α-turmerone, β-turmerone, and, ar-turmerone.

8. A method of enhancing bioavailability of a constituent in plasma, the method comprising administering the composition of claim 1, the constituent selected from the group consisting of curcumin, demethoxycurcumin, bisdemethoxycurcumin, α-turmerone, β-turmerone, and, ar-turmerone.

9. A method of preparing the composition of claim 1, the method comprising:
suspending a powdered extract of curcuminoid mixture in water to form a suspension, adding essential oil of turmeric to the suspension to obtain a mixture,
pulverizing the mixture to obtain a slurry,
stripping water from the slurry to obtain a powder comprising the composition of claim 1, and, wherein the essential oil of turmeric comprises about 30% to about 80% α-turmerone.

10. The method of claim 9, wherein the powdered extract curcuminoid mixture is prepared by a method comprising:
drying and powdering turmeric rhizomes to form a powdered rhizome,
extracting the powdered rhizome with ethyl acetate at 78° C. to obtain a residue and a supernatant,
cooling the supernatant at 4° C. to obtain crystals and a liquid,
powdering the crystals to obtain powdered extract of curcuminoid mixture, wherein the curcuminoid mixture consists of curcumin, demethoxycurcumin, and, bisdemethoxycurcumin.

11. The method of claim 9, wherein the essential oil of turmeric is prepared by a method comprising:
flaking fresh rhizome of turmeric to obtain flaked turmeric rhizome,
distilling the flaked turmeric rhizome in water at 64° C. to obtain distilled flakes of turmeric, soaking the distilled flakes of turmeric in water to obtain soaked flakes,
heating the soaked flakes of turmeric to 97° C. to obtain the essential oil of turmeric comprising about 30% α-turmerone.

12. The method of claim 11, wherein the essential oil of turmeric is prepared by a method further comprising:
purifying the essential oil of turmeric comprising about 30% α-turmerone of claim 11 by silica chromatography column using 99% hexane elution followed by elution with 1% ethyl acetate to obtain the essential oil of turmeric comprising about 40% α-turmerone.

13. The method of claim 11, wherein the essential oil of turmeric is prepared by a method further comprising:
purifying the essential oil of turmeric comprising about 30% α-turmerone of claim 11 by silica chromatography column using 98.5% hexane elution followed by elution with 1.5% ethyl acetate to obtain the essential oil of turmeric comprising about 60% α-turmerone.

14. The method of claim 13, wherein the essential oil of turmeric is prepared by a method further comprising:
purifying the essential oil of turmeric comprising about 60% α-turmerone of claim 13 by silica chromatography column using 99% hexane elution followed by elution with 1% chloroform to obtain the essential oil of turmeric comprising about 80% α-turmerone.

15. A composition for inhibiting beta secretase activity,
the composition comprising a curcuminoid mixture and a combination, the combination comprising an essential oil of turmeric and an extract of sesame oil,
wherein the curcuminoid mixture comprises curcumin, demethoxycurcumin, and, bisdemethoxycurcumin,
wherein the essential oil of turmeric comprises about 30% to about 80% of α-turmerone,
wherein the extract of sesame oil comprises about 30% to about 80% sesamolin,
wherein a weight ratio of the curcuminoid mixture to the combination is about 70:30,
wherein a weight ratio of the essential oil of turmeric to the extract of sesame oil ranges from about 1:3 to about 8:1.

16. The composition of claim 15 further comprising a property of enhancing bioavailability of a constituent, wherein the constituent is selected from the group consisting of curcumin, demethoxycurcumin, bisdemethoxycurcumin, α-turmerone, β-turmerone, ar-turmerone, and, sesamolin.

17. A dosage form comprising the composition of claim 15, the dosage form selected from the group consisting of capsule, tablet, granule, sachet, powder, paste, infusion, ampoule, solution, suspension, emulsion, pills, and, cream.

18. A method for inhibiting beta secretase in brain, the method comprising administering the composition of claim 15.

19. A method of enhancing bioavailability of a constituent in brain, the method comprising administering the composition of claim 15, the constituent selected from the group consisting of curcumin, demethoxycurcumin, bisdemethoxycurcumin, α-turmerone, β-turmerone, ar-turmerone, and, sesamolin.

20. A method of enhancing bioavailability of a constituent in serum, the method comprising administering the composition of claim 15, the constituent selected from the group consisting of curcumin, demethoxycurcumin, bisdemethoxycurcumin, α-turmerone, β-turmerone, ar-turmerone, and, sesamolin.

21. A method of enhancing bioavailability of a constituent in plasma, the method comprising administering the composition of claim 15, the constituent selected from the group consisting of curcumin, demethoxycurcumin, bisdemethoxycurcumin, α-turmerone, β-turmerone, ar-turmerone, and, sesamolin.

22. A method of treating a central nervous system disorder, the method comprising administering the composition of claim 15, the central nervous system disorder selected from the group consisting of epilepsy, migraine, Huntington's disease, Alzheimer's disease, depression, Parkinson's disease, Tourette syndrome, dystonia, multiple sclerosis, meningitis, lupus, fibromyalgia, and, bipolar disorder.

23. A method of preparing the composition of claim 15, the method comprising:
suspending a powdered extract of curcuminoid mixture in water to form a suspension,
adding a combination to the suspension to obtain a mixture,
pulverizing the mixture to obtain a slurry,
stripping water from the slurry to obtain a powder comprising the composition of claim 15,
wherein the combination comprises an essential oil of turmeric, and, an extract of sesame oil.

24. The method of claim 23, wherein the powdered extract curcuminoid mixture is prepared by a method comprising:
drying and powdering turmeric rhizomes to form a powdered rhizome,
extracting the powdered rhizome with ethyl acetate at 78° C. to obtain a residue and a supernatant,
cooling the supernatant at 4° C. to obtain crystals and a liquid,
powdering the crystals to obtain powdered extract of curcuminoid mixture, wherein the curcuminoid mixture consists of curcumin, demethoxycurcumin, and, bisdemethoxycurcumin.

25. The method of claim 23, wherein the essential oil of turmeric is prepared by a method comprising:
flaking fresh rhizome of turmeric to obtain flaked turmeric rhizome,
distilling the flaked turmeric rhizome in water at 64° C. to obtain distilled flakes of turmeric, soaking the distilled flakes of turmeric in water to obtain soaked flakes,
heating the soaked flakes of turmeric to 97° C. to obtain an essential oil of turmeric comprising about 30% α-turmerone.

26. The method of claim 25, wherein the essential oil of turmeric is prepared by a method further comprising:
purifying the essential oil of turmeric comprising about 30% α-turmerone by silica chromatography column using 99% hexane elution followed by elution with 1% ethyl acetate to obtain an essential oil of turmeric comprising about 40% α-turmerone.

27. The method of claim 25, wherein the essential oil of turmeric is prepared by a method further comprising:
purifying the essential oil of turmeric comprising about 30% α-turmerone by silica chromatography column using 98.5% hexane elution followed by elution with 1.5% ethyl acetate to obtain an essential oil of turmeric comprising about 60% α-turmerone.

28. The method of claim 27, wherein the essential oil of turmeric is prepared by a method further comprising:
purifying the essential oil of turmeric comprising about 60% α-turmerone by silica chromatography column using 99% hexane elution followed by elution with 1% chloroform to obtain an essential oil of turmeric comprising about 80% α-turmerone.

29. The method of claim 23, wherein the extract of sesame oil is prepared by a method comprising:
extracting a first extract of sesame oil from sesame seed,
adding n-hexane to the first extract of sesame oil to obtain a mixture,
passing the mixture through an adsorption column and obtaining a first hexane elute and a first ethyl acetate elute,
evaporating the first ethyl acetate elute to obtain a second extract of sesame oil,
allowing the second extract of sesame oil to set overnight to form a precipitate,
filtering the precipitate, wherein the precipitate comprises crystals having about 20% sesamolin,
loading the crystals having about 20% sesamolin on a silica chromatography column,
eluting the chromatographic column with 85% hexane in ethyl acetate to obtain a second hexane elute and a second ethyl acetate elute,
loading the second ethyl acetate elute onto a silica chromatography column and eluting with (1) first with 91% hexane to obtain a 91% hexane elute, followed by a second elution with 9% ethyl acetate to obtain a 9% ethyl acetate elute which comprises a third extract of sesame oil, wherein the third extract of sesame oil comprises about 60% sesamolin, loading the third extract of sesame oil onto a silica chromatography column and eluting with 93% hexane and 7% ethyl acetate to obtain a 93% hexane elute and a 7% ethyl acetate elute, wherein the 7% ethyl acetate elute comprises a fourth extract of sesame oil, wherein the fourth extract of sesame oil comprises about 80% sesamolin,
(2) eluting with a gradient of 92% hexane and 8% ethyl acetate to obtain a 92% hexane elute and an 8% ethyl acetate elute, wherein the 8% ethyl acetate elute comprises a fifth extract of sesame oil, wherein the fifth extract of sesame oil comprises about 55% sesamolin.

30. A composition for inhibiting beta secretase activity,
wherein the composition comprises an essential oil of turmeric and an extract of sesame oil.

31. The composition of claim 30, wherein the essential oil of turmeric comprises about 30% to about 80% of α-turmerone.

32. The composition of claim 30, wherein the extract of sesame oil comprises about 30% to about 80% sesamolin.

33. The composition of claim 30, wherein a weight ratio of the essential oil of turmeric to the extract of sesame oil ranges from about 1:3 to about 8:1.

34. The composition of claim 30 further comprising a property of enhancing bioavailability of a constituent, wherein the constituent is selected from the group consisting of α-turmerone, β-turmerone, ar-turmerone, and, sesamolin.

35. A dosage form comprising the composition of claim 30, the dosage form selected from the group consisting of capsule, tablet, granule, sachet, powder, paste, infusion, ampoule, solution, suspension, emulsion, pills, and, cream.

36. A method of inhibiting beta secretase activity in brain, the method comprising administering the composition of claim 30.

37. A method for enhancing the bioavailability of a constituent in brain, the method comprising administering the composition of claim 30, the constituent selected from the group consisting of α-turmerone, β-turmerone, ar-turmerone, and, sesamolin.

38. A method for enhancing the bioavailability of a constituent in serum, the method comprising administering the composition of claim 30, the constituent selected from the group consisting of α-turmerone, β-turmerone, ar-turmerone, and, sesamolin.

39. A method for enhancing the bioavailability of a constituent in plasma, the method comprising administering the composition of claim 30, the constituent selected from the group consisting of α-turmerone, 3-turmerone, ar-turmerone, and, sesamolin.

40. A method of treating a central nervous system disorder, the method comprising administering the composition of claim 30, the central nervous system disorder selected from the group consisting of epilepsy, migraine, Huntington's disease, Alzheimer's disease, depression, Parkinson's disease, Tourette syndrome, dystonia, multiple sclerosis, meningitis, lupus, fibromyalgia, and, bipolar disorder.

41. A method of preparing a composition for inhibiting beta secretase activity, the composition comprising an essential oil of turmeric and an extract of sesame oil, the method comprising:
mixing essential oil of turmeric and extract of sesame oil to obtain a first mixture,
warming the first mixture to 65° C. to obtain a warm mixture,
blending the warm mixture to obtain a blend of the composition.

42. The method of claim 41, wherein the essential oil of turmeric is prepared by a method comprising:
flaking fresh turmeric rhizome to obtain a flaked turmeric rhizome,
distilling the flaked turmeric rhizome with water at a temperature of 64° C. to obtained a distilled flaked turmeric rhizome,
soaking the distilled flaked turmeric rhizome in water to obtain a soaked flake of turmeric rhizome,
heating the soaked flake of turmeric rhizome to about 97° C. to obtain a first essential oil of turmeric, wherein the first essential oil of turmeric comprises about 30% α-turmerone.

43. The method of claim 42, wherein the essential oil of turmeric is prepared by a method further comprising:
purifying the essential oil of turmeric comprising about 30% α-turmerone of claim 42 by silica chromatography column using 99% hexane elution followed by elution with 1% ethyl acetate to obtain the essential oil of turmeric comprising about 40% α-turmerone.

44. The method of claim 42, wherein the essential oil of turmeric is prepared by a method further comprising:
purifying the essential oil of turmeric comprising about 30% α-turmerone of claim 42 by silica chromatography column using 98.5% hexane elution followed by elution with 1.5% ethyl acetate to obtain the essential oil of turmeric comprising about 60% α-turmerone.

45. The method of claim 44, wherein the essential oil of turmeric is prepared by a method further comprising:
purifying the essential oil of turmeric comprising about 60% α-turmerone of claim 44 by silica chromatography column using 99% hexane elution followed by elution with 1% chloroform to obtain the essential oil of turmeric comprising about 80% α-turmerone.

46. The method of claim 41, wherein the extract of sesame oil is prepared by a method comprising:
extracting a first extract of sesame oil from sesame seed,
adding n-hexane to the first extract of sesame oil to obtain a mixture,
passing the mixture through an adsorption column and obtaining a first hexane elute and a first ethyl acetate elute,
evaporating the first ethyl acetate elute to obtain a second extract of sesame oil,
allowing the second extract of sesame oil to set overnight to form a precipitate,
filtering the precipitate, wherein the precipitate comprises crystals having about 20% sesamolin.

47. The method of claim 46 further comprising:
loading the crystals having about 20% sesamolin of claim 46 on a silica chromatography column,
eluting the silica chromatography column with 85% hexane in ethyl acetate to obtain a second hexane elute, and, a second ethyl acetate elute, wherein the second ethyl acetate elute comprises 40% sesamolin,
loading the second ethyl acetate elute onto a silica chromatography column and eluting with (1) 91% hexane to obtain a 91% hexane elute, followed by an elution with 9% ethyl acetate to obtain a 9% ethyl acetate, wherein the 9% ethyl acetate elute comprises a third extract of sesame oil, wherein the third extract of sesame oil comprises 60% sesamolin.

48. The method of claim 47 further comprising:
loading the third extract of sesame oil of claim 47 onto a silica chromatography column and eluting with 93% hexane and 7% ethyl acetate to obtain a 93% hexane elute and a 7% ethyl acetate elute, wherein the 7% ethyl acetate elute comprises a fourth extract of sesame oil, wherein the fourth extract of sesame oil comprises about 80% sesamolin.

49. The method of claim 47 further comprising:
loading the second ethyl acetate elute comprising about 40%/6 sesamolin of claim 47 on a silica chromatography column,
eluting with a gradient of 92% hexane and 8% ethyl acetate to obtain a 92% hexane elute and an 8% ethyl acetate elute,
wherein the 8% ethyl acetate elute comprises a fifth extract of sesame oil, and wherein the fifth extract of sesame oil comprises about 55% sesamolin.
